(19) United States
(12) Patent Application Publication
(10) Pub. No.: US 2024/0358544 A1
(43) Pub. Date: Oct. 31, 2024

(54) METHOD OF TREATING VISION LOSS IN A HUMAN SUBJECT WITH PHOTOREACTIVE COMPOUNDS

(71) Applicant: KIORA PHARMACEUTICALS PTY LTD, Camberwell, VIC (AU)

(72) Inventors: Brian STREM, Encinitas, CA (US); Stefan SPERL, Wien (AT); Eric DANIELS, Camberwell, VIC (AU)

(21) Appl. No.: 18/647,758

(22) Filed: Apr. 26, 2024

Related U.S. Application Data
(60) Provisional application No. 63/498,708, filed on Apr. 27, 2023.

(51) Int. Cl. A61F 9/00; A61P 27/10

(52) U.S. Cl. CPC A61F 9/0017; A61P 27/10

(57) ABSTRACT

The present disclosure provides a method of treating a human subject suffering from a retinal disorder, the method comprising administering to the eye of the subject via intravitreal injection a composition comprising a compound of formula (I) as described herein.

### FIELD OF THE INVENTION

The present disclosure provides a method of treating a human subject suffering from a retinal disorder, the method comprising administering to the eye of the subject via intravitreal injection a composition comprising a compound of formula (I) as described herein.

### BACKGROUND

Certain photochromic azobenzene compounds exhibit photoswitch behavior, whereby the compounds undergo photoisomerization, changing the length and geometry of the compounds. Such compounds can interact with proteins, and thereby alter protein function when the compounds undergo photoisomerization. These compounds have been shown in vitro to confer light-sensitivity to degenerated retinas by interaction with membrane proteins in retinal ganglion cells and upstream neurons.

### SUMMARY OF THE INVENTION

In some embodiments, the present disclosure provides a method of treating a human subject suffering from a retinal disorder, the method comprising administering to the eye of the subject via intravitreal injection a composition comprising a compound of formula (I):

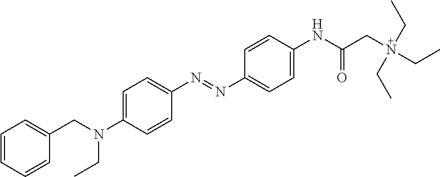

or a pharmaceutically acceptable salt thereof, wherein the azo (N═N) bond in the structure may be either cis or trans.

In some embodiments, the retinal disorder is caused by a genetic eye disease. In some embodiments, the genetic eye disease is retinitis pigmentosa. In some embodiments, the retinal disorder is age-related macular degeneration. In some embodiments, the retinal disorder is choroideremia.

In some embodiments, the compound of formula (I) is administered to both eyes of the subject. In some embodiments, the compound of formula (I) is only administered to one eye of the subject. In some embodiments, the administering to one eye of the subject provides a treatment effect in both eyes of the subject.

In some embodiments, the administering to one eye is a first administration, and wherein the method further comprises a second administration, wherein the second administration comprises administering the compound of formula (I) to contralateral eye of the subject about 1 week to about 2 months after the first administration. In some embodiments, the administering to one eye is a first administration, and wherein the method further comprises a second administration, wherein the second administration comprises administering the compound of formula (I) to the same eye of the subject about 1 week to about 2 months after the first administration. In some embodiments, the administering to one eye is a first administration, and wherein the method further comprises a second administration, wherein the second administration comprises administering the compound of formula (I) to contralateral eye of the subject about 3 weeks to about 5 weeks after the first administration. In some embodiments, the administering to one eye is a first administration, and wherein the method further comprises a second administration, wherein the second administration comprises administering the compound of formula (I) to the same eye of the subject about 3 weeks to about 5 weeks after the first administration. In some embodiments, the administering to one eye is a first administration, and wherein the method further comprises a second administration, wherein the second administration comprises administering the compound of formula (I) to contralateral eye of the subject about 1 month after the first administration. In some embodiments, the administering to one eye is a first administration, and wherein the method further comprises a second administration, wherein the second administration comprises administering the compound of formula (I) to the same eye of the subject about 1 month to about 2 months after the first administration.

In some embodiments, the treating a human subject comprises increasing the subject's ability to detect light and contrast as measured by a light response test. In some embodiments, the treating a human subject comprises stimulating striate (V1) and non-striate (V2 and/or V3) cortical activity in the subject as measured by Functional MRI.

In some embodiments, the treating a human subject comprises an increase in tolerance of glare and photosensitivity in the subject. In some embodiments, the treating of a human subject comprises increasing measures of functional vision. In some embodiments, the treating of a human subject reduces the non-specific, non-stimulus generated, random electrical activity of the retina.

In some embodiments, the composition further comprises a cyclodextrin. In some embodiments, a molar ratio of cyclodextrin to the compound of formula (I) in the composition is about 20:1 to about 1:1. In some embodiments, a molar ratio of cyclodextrin to the compound of formula (I) in the composition is about 15:1 to about 3:1. In some embodiments, a molar ratio of cyclodextrin to the compound of formula (I) in the composition is about 5:1.

In some embodiments, the concentration of the compound of formula (I) in the composition is from about 0.1 mM to about 10 mM. In some embodiments, the concentration of the compound of formula (I) in the composition is from about 0.2 mM to about 5 mM. In some embodiments, the human subject is administered about 1 μg to about 100 μg of the compound of formula (I) to a single eye. In some embodiments, the human subject is administered about 7.5 μg to about 50 μg of the compound of formula (I) to a single eye.

In some embodiments, the present disclosure provides a method of treating a human subject suffering from a retinal disorder, the method comprising: (a) a first administration to one eye of the subject via intravitreal injection a composition comprising a compound of formula (I); and a second administration to the contralateral eye of the subject via intravitreal injection a composition comprising a compound of formula (I); or (b) a first administration to one eye of the subject via intravitreal injection a composition comprising a compound of formula (I); and a second administration to the same eye of the subject via intravitreal injection a composition comprising a compound of formula (I); wherein the compound of formula (I) has the structure:

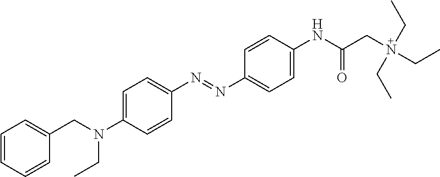

or a pharmaceutically acceptable salt thereof, wherein the azo bond in the structure may be either cis or trans.

In some embodiments, the second administration to the contralateral eye is simultaneous or substantially simultaneous with the first administration, or immediately following the first administration. In some embodiments, the second administration to the contralateral eye is about 3 weeks to about 5 weeks after the first administration. In some embodiments, the second administration to the same eye is about 3 weeks to about 5 weeks after the first administration.

In some embodiments, the present disclosure provides a method of increasing the tolerance from glare and photosensitivity in a human subject suffering from a retinal disorder, the method comprising: (a) a first administration to one eye of the subject via intravitreal injection a composition comprising a compound of formula (I); and a second administration to the contralateral eye of the subject via intravitreal injection a composition comprising a compound of formula (I); or (b) a first administration to one eye of the subject via intravitreal injection a composition comprising a compound of formula (I); and a second administration to the same eye of the subject via intravitreal injection a composition comprising a compound of formula (I); wherein the compound of formula (I) has the structure:

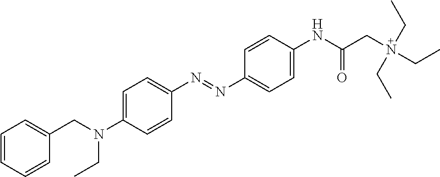

or a pharmaceutically acceptable salt thereof, wherein the azo bond in the structure may be either cis or trans.

### BRIEF DESCRIPTION OF THE DRAWINGS

The following drawings form part of the present specification and are included to further demonstrate exemplary embodiments of certain aspects of the present disclosure.

FIG. 1 shows a clinical study design as described in Example 1.

FIGS. 2A-2C show results of an intensity and contrast assessment of a subject administered with KIO-301 (Subject 1-01). FIG. 2A shows the intensity and contrast assessment results for the subject's right eye (OD). FIG. 2B shows the intensity and contrast assessment results for the subject's left eye (OS). FIG. 2C shows the subject's Light Response Index relative to light intensity.

FIGS. 3A and 3B show results of a kinetic visual field assessment of Subject 1-01. FIG. 3A shows the resulting perimeter chart with filled circles indicating the baseline and hatched circles indicating results at 1 month post-injection. FIG. 3B shows a summary of the total horizontal and vertical visual field degrees for the right and left eyes, at baseline and 1 month post-injection.

FIG. 4 shows results of a window location assessment of Subject 1-01, conducted over 30 days. OD: right eye; OS: left eye; OU: both eyes.

FIG. 5 shows results of a functional MRI performed on Subject 1-01, at baseline and Days 2, 14, and 28 post-injection.

FIG. 6 shows results of a functional MRI performed on Subject 1-01 and subjected to checkerboard stimuli.

FIG. 7 shows results of a quality of life survey (VFQ-25) responses by Subject 1-01 at baseline and Day 29 post-injection.

FIGS. 8A-8C show results of an intensity and contrast assessment of a subject administered with KIO-301 (Subject 1-02). FIG. 2A shows the intensity and contrast assessment results for the subject's right eye (OD). FIG. 2B shows the intensity and contrast assessment results for the subject's left eye (OS). FIG. 2C shows the intensity and contrast assessment results for both eyes of the subject (OU).

FIGS. 9A and 9B show results of a kinetic visual field assessment of Subject 1-02. FIG. 9A shows the resulting perimeter chart. FIG. 3B shows a summary of the total horizontal and vertical visual field degrees for the right and left eyes, at baseline and 1 week post-injection.

FIG. 10 shows results of a functional MRI performed on Subject 1-02, at baseline and Days 2, 14, and 28 post-injection.

### DETAILED DESCRIPTION

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or “about” can mean rounded to the nearest significant digit.

As used herein, “between” is a range inclusive of the ends of the range. For example, a number between x and y explicitly includes the numbers x and y and any numbers that fall within x and y.

As used herein, the term “active agent” includes any agent, drug, compound, composition, or other substance that may be used on, or administered to a subject, e.g., a human or animal subject, for any purpose, including therapeutic, pharmaceutical, pharmacological, diagnostic, cosmetic, and prophylactic agents and immunomodulators. The term “active agent” may be used interchangeably with the terms “drug,” “pharmaceutical,” “medicament,” “drug substance,” and “therapeutic.” In some embodiments, the active agent of the present disclosure comprises a compound of formula (I).

In some embodiments, the compositions described herein are “pharmaceutically acceptable.” The term “pharmaceutically acceptable” when referring to the compositions described herein or the excipients, carriers, diluents, or ingredients described herein, means that the composition and/or components thereof are suitable for use in humans and/or animals without excessive adverse side effects (such as toxicity, irritation, and allergies), that is, with a reasonable benefit/risk ratio.

The term “side effect” or “adverse event” means physiological disease and/or adverse conditions attributable to a treatment other than the desired effects.

In some embodiments, the compositions herein are effective for the treatments described herein. The term “efficacy” means the ability to produce a desired effect. The term “effective amount” refers to an amount of a therapeutic agent (e.g., a compound of formula (I) described herein) to treat, alleviate or prevent a target disease or condition (e.g., a retinal disorder described herein), or an amount that exhibits a detectable therapeutic or preventive effect. The exact effective amount for a subject may depend on the subject's size and health, the nature and extent of the condition, and the chosen therapeutic agent and/or combination of therapeutic agents.

A “therapeutically effective amount” is the amount of compound, e.g., a compound of formula (I) as described herein, which achieves a therapeutic effect by inhibiting a condition or disorder in a patient, e.g., retinitis pigmentosa (RP). A therapeutically effective amount may be an amount which relieves to some extent one or more symptoms of a condition or disorder in a patient; returns to normal either partially or completely one or more physiological or biochemical parameters associated with or causative of the condition or disorder; and/or reduces the likelihood of the onset of the condition of disorder.

The term “administration” or “administering” refers to routes of introducing a compound or composition provided herein to an individual to perform its intended function. An example of a route of administration that can be used includes, but is not limited to, parenteral administration, such as intravitreal injection.

The term “subject” means any subject, particularly a mammalian subject, in need of treatment, e.g., with a composition comprising a compound of formula (I). In some embodiments, the term “subject” refers to a human subject. As used herein, a “subject in need thereof” refers to the subject for whom it is desirable to treat, e.g., a subject having retinal disorder described herein. In some embodiments, the term “subject in need thereof” can refer to a subject at high risk for suffering from a condition suitable to treatment with the composition comprising a compound of formula (I) as described herein, independently of whether the subject has physical manifestations of such condition.

### Treatment of Retinal Disorder

In some embodiments, the disclosure provides a method of treating a human subject suffering from a retinal disorder, the method comprising administering to the eye of the subject via intravitreal injection a composition comprising a compound of formula (I):

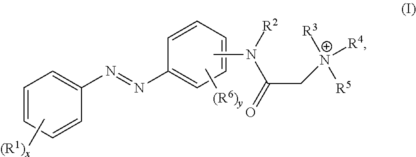

or a pharmaceutically acceptable salt thereof, wherein R¹, R², R³, R⁴, R⁵, R⁶, x, and y are as described herein.

In some embodiments, the disclosure provides a method of treating a human subject suffering from a retinal disorder, the method comprising: (a) a first administration to one eye of the subject via intravitreal injection a composition comprising a compound of formula (I); and a second administration to the contralateral eye of the subject via intravitreal injection a composition comprising a compound of formula (I); or (b) a first administration to one eye of the subject via intravitreal injection a composition comprising a compound of formula (I); and a second administration to the same eye of the subject via intravitreal injection a composition comprising a compound of formula (I); wherein the compound of formula (I) has the structure:

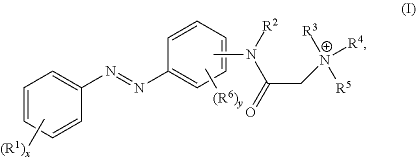

or a pharmaceutically acceptable salt thereof, wherein R¹, R², R³, R⁴, R⁵, R⁶, x, and y are as described herein.

In some embodiments, the disclosure provides a method of increasing the tolerance from glare and photosensitivity in a human subject suffering from a retinal disorder, the method comprising: (a) a first administration to one eye of the subject via intravitreal injection a composition comprising a compound of formula (I); and a second administration to the contralateral eye of the subject via intravitreal injection a composition comprising a compound of formula (I); or (b) a first administration to one eye of the subject via intravitreal injection a composition comprising a compound of formula (I); and a second administration to the same eye of the subject via intravitreal injection a composition comprising a compound of formula (I); wherein the compound of formula (I) has the structure:

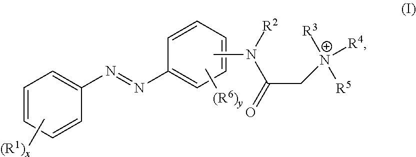

or a pharmaceutically acceptable salt thereof, wherein R¹, R², R³, R⁴, R⁵, R⁶, x, and y are as described herein.

In some embodiments, the compound of formula (I) has the structure:

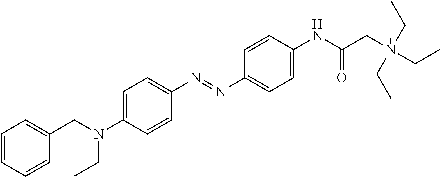

or a pharmaceutically acceptable salt thereof, wherein the azo (N═N) bond in the structure may be either cis or trans.

It was surprisingly discovered that compositions comprising the compounds described herein, e.g., compounds of formula (I), are effective for treating retinal disorders in humans. In some embodiments, the compositions described herein confer light sensitivity to human retinal pigment epithelial cells and cells disposed in the neurosensory retina, for example, photoreceptor cells and Mueller cells. Throughout the present disclosure, any reference to a “subject” is meant to encompass a human subject.

In some embodiments, the retinal disorder is diabetic retinopathy, age-related macular degeneration (AMD or ARMD) (wet form), dry AMD, retinopathy of prematurity, retinitis pigmentosa, choroideremia, and/or glaucoma, including open-angle glaucoma (e.g., primary open-angle glaucoma), angle-closure glaucoma, and secondary glaucomas (e.g., pigmentary glaucoma, pseudoexfoliative glaucoma, and glaucomas resulting from trauma and inflammatory diseases). In some embodiments, the retinal disorder is AMD, e.g., wet AMD or dry AMD. In some embodiments, the retinal disorder is retinitis pigmentosa. In some embodiments, the retinal disorder is choroideremia. In some embodiments, the retinal disorder has been diagnosed by a qualified medical professional.

In some embodiments, the retinal disorder is retinal detachment, age-related or other maculopathies, photic retinopathies, surgery-induced retinopathies, toxic retinopathies, retinopathy of prematurity, retinopathies due to trauma or penetrating lesions of the eye, inherited retinal degenerations, surgery-induced retinopathies, toxic retinopathies, and/or retinopathies due to trauma or penetrating lesions of the eye.

In some embodiments, the retinal disorder is caused by a genetic eye disease. Exemplary genetic eye diseases include, but are not limited to, Bardet-Biedl syndrome (autosomal recessive), congenital amaurosis (autosomal recessive), cone or cone-rod dystrophy (autosomal dominant and X-linked forms), congenital stationary night blindness (autosomal dominant, autosomal recessive and X-linked forms), macular degeneration (autosomal dominant and autosomal recessive forms), optic atrophy, autosomal dominant and X-linked forms), retinitis pigmentosa (autosomal dominant, autosomal recessive and X-linked forms), syndromic or systemic retinopathy (autosomal dominant, autosomal recessive and X-linked forms), Usher syndrome (autosomal recessive), and choroideremia (X-linked). In some embodiments, the genetic eye disease is retinitis pigmentosa. In some embodiments, the genetic eye disease is choroideremia.

The compositions described herein can be administered to the eye through a variety of routes, e.g., as described herein. A composition may be delivered intraocularly, by topical application to the eye or by intraocular injection into, for example, the vitreous or subretinal (interphotoreceptor) space. Alternatively, a composition may be delivered locally by insertion or injection into the tissue surrounding the eye. A composition may be delivered systemically through an oral route or by subcutaneous, intravenous or intramuscular injection. Alternatively, a composition may be delivered by means of a catheter or by means of an implant, wherein such an implant is made of a porous, non-porous or gelatinous material, including membranes such as silastic membranes or fibers, biodegradable polymers, or proteinaceous material. A composition can be administered prior to the onset of the condition, to prevent its occurrence, for example, during surgery on the eye, or immediately after the onset of the pathological condition or during the occurrence of an acute or protracted condition. In some embodiments, the composition comprising the compound of formula (I) described herein is administered via intravitreal injection (IVT).

In some embodiments, the compound of formula (I) is administered to both eyes of the human subject. In some embodiments, both eyes of the human subject are administered simultaneously or substantially simultaneously. In some embodiments, one of the human subject is administered immediately following administration to the other eye. In some embodiments, one eye of the human subject is administered within 1 hour, within 2 hours, within 4 hours, within 6 hours, within 8 hours, within 12 hours, within 18 hours, or within 24 hours of administration to the other eye. In some embodiments, one eye of the human subject is administered within about 1 day to about 3 months, or about 1 week to about 2 months, or about 2 weeks to about 6 weeks, or about 3 weeks to about 5 weeks, or about 1 month of administration to the other eye. In some embodiments, both eyes of the human subject are administered with the compound of formula (I) during a single clinical or hospital visit. In some embodiments, each eye of the human subject is administered with the compound of formula (I) during separate clinical or hospital visits.

In some embodiments, the compound of formula (I) is only administered to one eye of the human subject. It was surprisingly discovered that administration of the compound of formula (I) in one eye of the subject provided a treatment effect in both eyes of the subject. In some embodiments, the treatment effect comprises one or more of: increasing the subject's ability to detect light and contrast as measured by a light response test; increasing the subject's visual field; increasing the subject's ability to determine direction of light; stimulating striate (V1) and/or non-striate (V2 and/or V3) cortical activity in the subject as measured by Functional MRI; increasing the subject's tolerance of glare and photosensitivity; increasing measures of the subject's functional vision, e.g., via a Functional Vision Assessment (FVA); and reducing non-specific, non-stimulus generated, random electrical activity of the retina (also known as “ringing” of the eye as perceived by the brain).

Functional magnetic resonance imaging or functional MRI measures brain activity by detecting changes associated with blood flow. In some embodiments, functional MRI can rely on the fact that cerebral blood flow and neuronal activation are coupled, wherein when an area of the brain is in use, blood flow to that region also increases. In some embodiments, functional MRI uses the blood-oxygen-level dependent (BOLD) contrast. See, e.g., S. A. Huettel, et al (2009), page 4 and page 26, Functional Magnetic Resonance Imaging (2 ed.), Massachusetts: Sinauer. In some embodiments, the functional MRI can use arterial spin labeling and diffusion MRI. See, e.g., J. A. Detre, John A. et al. (May 2012), Journal of Magnetic Resonance Imaging. 35 (5): 1026-1037. Diffusion MRI is similar to BOLD fMRI but provides contrast based on the magnitude of diffusion of water molecules in the brain. Functional Vision Assessment (FVA) refers to any of the standardized tests that evaluate the functioning of how a subject uses their remaining vision across a variety of familiar and unfamiliar environments. In some embodiments, the FVA will investigate how the subject uses his vision for (i) near tasks, closer than 16 inches; (ii) intermediate tasks, 16 inches to 3 feet; and (iii) distance tasks, more than 3 feet away. In some embodiments, the FVA is conducted by a trained specialist. In some embodiments, the FVA can include one or more of the following: (i) how clear and sharp the subject's vision is, (ii) visual field, or the area the subject sees to the sides, above, and below (known as the peripheral area of vision), (iii) contrast sensitivity, or the ability of the subject to detect differences in grayness and between objects and their background, (iv) color vision, or the ability to detect different colors and also hues within a color, (v) light sensitivity, or response to light (sunlight or artificial light).

In some embodiments, the methods provided herein comprise administering the compound of formula (I) to one eye of the human subject, i.e., a first administration, followed by a second administration of the compound of formula (I) to the same eye or a contralateral eye of the human subject. In some embodiments, the second administration is simultaneous or substantially simultaneous with the first administration, or immediately following the first administration. In some embodiments, the second administration comprises administering the compound of formula (I) to the contralateral eye of the subject simultaneous or substantially simultaneous with the first administration, or immediately following the first administration. In some embodiments, the second administration comprises administering the compound of formula (I) to the contralateral eye of the subject on the same day as the first administration; or about 1 day to about 3 months, or about 1 week to about 2 months, or about 2 weeks to about 6 weeks, or about 3 weeks to about 5 weeks, or about 1 month after the first administration. In some embodiments, the second administration comprises administering the compound of formula (I) to the contralateral eye of the subject about 1 week to about 2 months after the first administration. In some embodiments, the second administration comprises administering the compound of formula (I) to the contralateral eye of the subject about 3 weeks to 5 weeks after the first administration. In some embodiments, the second administration comprises administering the compound of formula (I) to the contralateral eye of the subject about 1 month after the first administration.

In some embodiments, the intravitreal administration of the compounds of formula (I) can continue every week to every 2 months for greater than 1, greater than 2, greater than 3, greater than 4, greater than 5, greater than 6 greater than 7, greater than 8, greater than 9, or greater than 10 administrations. Thus, e.g., the compounds of formula (I) can be administered every week to every month for greater than 6 months, greater than 12 months, greater than 18 months, greater than 2 years, greater than 5 years or for the remainder of the life of the subject. In some embodiments, the intravitreal administration of the compounds of formula (I) can continue every 3 weeks to every 5 weeks for greater than 1, greater than 2, greater than 3, greater than 4, greater than 5, greater than 6 greater than 7, greater than 8, greater than 9, or greater than 10 administrations. Thus, e.g., the compounds of formula (I) can be administered every 3 weeks to every 5 weeks for greater than 6 months, greater than 12 months, greater than 18 months, greater than 2 years, greater than 5 years or for the remainder of the life of the subject. In some embodiments, the compounds of formula (I) are administered in perpetuity for the life of the human subject. In some embodiments, the compounds of formula (I) are administered “as needed” by the patient or “as desired” by the human subject in perpetuity for the life of the subject.

In some embodiments, the second administration comprises administering the compound of formula (I) to the same eye of the subject simultaneously or substantially simultaneously with the first administration, or immediately following the first administration. In some embodiments, the second administration comprises administering the compound of formula (I) to the same eye of the subject on the same day as the first administration, or about 1 day to about 3 months, or about 1 week to about 2 months, or about 2 weeks to about 6 weeks, or about 3 weeks to about 5 weeks, or about 1 month after the first administration. In some embodiments, the second administration comprises administering the compound of formula (I) to the same eye of the subject about 1 week to about 2 months after the first administration. In some embodiments, the second administration comprises administering the compound of formula (I) to the same eye of the subject about 3 weeks to 5 weeks after the first administration. In some embodiments, the second administration comprises administering the compound of formula (I) to the same eye of the subject about 1 month after the first administration. In some embodiments, a second administration to the same eye as the first administration provides a treatment effect in both eyes of the subject.

In some embodiments, treating the subject suffering from a retinal disorder comprises one or more of: increasing the subject's ability to detect light and contrast as measured by a light response test; increasing the subject's visual field; increasing the subject's ability to determine direction of light; stimulating striate (V1) and/or non-striate (V2 and/or V3) cortical activity in the subject as measured by Functional MRI; increasing the subject's tolerance of glare and photosensitivity; increasing measures of the subject's functional vision, e.g., via a Functional Vision Assessment (FVA); and reducing non-specific, non-stimulus generated, random electrical activity of the retina (also known as “ringing” of the eye as perceived by the brain).

In some embodiments, the treating comprises increasing the subject's ability to detect light and contrast (i.e., the difference between light and dark perception) at various intensities of light. An exemplary method of assessing the subject's ability to detect light and contrast includes presenting to the subject a series of visual stimuli (e.g., light flashes) at various intensities and asking the subject to acknowledge when the stimuli is perceived. In some embodiments, the light intensity tested on the subject ranges from about 10¹³ to about 10¹⁷ photons/cm²·s. In some embodiments, the subject's percent accuracy for identifying the visual stimuli by one or both eyes is increased by at least 1.5-fold, at least 2-fold, at least 3-fold, at least 4-fold, or at least 5-fold at 2 days after the treatment. In some embodiments, the subject's percent accuracy for identifying the visual stimuli by one or both eyes is increased by at least 1.5-fold, at least 2-fold, at least 3-fold, at least 4-fold, or at least 5-fold at 10 days after the treatment. In some embodiments, the subject's percent accuracy for identifying the visual stimuli by one or both eyes is increased by at least 1.5-fold, at least 2-fold, at least 3-fold, at least 4-fold, or at least 5-fold at 30 days after the treatment. In some embodiments, the treatment comprises administering the composition comprising a compound of formula (I) into one eye of the subject as described herein, wherein the increase in the subject's ability to detect light and contrast is in both eyes of the subject. In some embodiments, the treatment comprises administering the composition comprising a compound of formula (I) into both eyes of the subject as described herein.

In some embodiments, the treating comprises increasing the subject's visual field, e.g., increase in total horizontal degrees and/or total vertical degrees of vision. In some embodiments, the subject's total horizontal degrees and/or total vertical degrees of vision is increased by at least 1.5-fold, at least 2-fold, at least 3-fold, at least 4-fold, at least 5-fold, or at least 10-fold at about 2 to about 30 days after the treatment, e.g., about 2 days, about 7 days, about 10 days, about 15 days, or about 30 days after the treatment. In some embodiments, the treatment comprises administering the composition comprising a compound of formula (I) into one eye of the subject as described herein, wherein the increase in the subject's visual field is in both eyes of the subject. In some embodiments, the treatment comprises administering the composition comprising a compound of formula (I) into both eyes of the subject as described herein.

In some embodiments, the treating comprises increasing the subject's ability to determine direction of light. An exemplary method of assessing the subject's ability to determine direction of light includes asking the subject to identify the location of a window, which tests the subject's ability to determine direction. In some embodiments, the subject's percent accuracy for identifying the window location by one or both eyes is increased by at least 1.5-fold, at least 2-fold, at least 3-fold, at least 4-fold, at least 5-fold, or at least 10-fold at 2 days after the treatment. In some embodiments, the subject's percent accuracy for identifying the window location by one or both eyes is increased by at least 1.5-fold, at least 2-fold, at least 3-fold, at least 4-fold, or at least 5-fold at 10 days after the treatment. In some embodiments, the subject's percent accuracy for identifying the window location by one or both eyes is increased by at least 1.5-fold, at least 2-fold, at least 3-fold, at least 4-fold, or at least 5-fold at 30 days after the treatment. In some embodiments, the treatment comprises administering the composition comprising a compound of formula (I) into one eye of the subject as described herein, wherein the increase in the subject's ability to determine direction of light is in both eyes of the subject. In some embodiments, the treatment comprises administering the composition comprising a compound of formula (I) into both eyes of the subject as described herein.

In some embodiments, the treating comprises increasing the visual cortex activity of the subject. In some embodiments, a visual stimulus, e.g., a checkerboard, is presented to the subject, and neural activity of the subject is measured using Functional MRI. In some embodiments, the treating comprises stimulating striate (V1) and non-striate (V2 and/or V3) cortical activity in the subject as measured by Functional MRI. V1 is the primary visual cortex and is the first stage of visual processing. V2 is the secondary visual cortex and receives integrated information from V1 and has feedforward connections with V3-V5. V3 refers to the third visual complex, which may play a role in processing motion and color sensitivity. See, e.g., Huff et al., Neuroanatomy, Visual Cortex. [Updated 2022 Jul. 25]. In: StatPearls [Internet]. Treasure Island (FL): StatPearls Publishing; 2023 January. In some embodiments, the subject's V1, V2, and/or V3 cortical activity is stimulated about 2 to about 30 days after the treatment, e.g., about 2 days, about 7 days, about 10 days, about 15 days, or about 30 days after the treatment. In some embodiments, the treating comprises administering the composition comprising a compound of formula (I) into one or both eyes of the subject as described herein.

In some embodiments, the treating comprises increasing the subject's tolerance of glare and photosensitivity while improving the subject's vision, e.g., increasing the subject's ability to detect light and contrast; increasing the subject's visual field; and/or increasing the subject's ability to determine direction of light as described herein. Glare sensitivity is a loss of visual acuity in bright lighting, such as when near a bright light source or outdoors in bright sunlight. Subjects suffering from glare sensitivity are unable to see the separate contours of brightly lit objects, and their surroundings may merge into a “wall” of bright white. Unexpectedly, the compounds of formula (I) described herein, which are photoreactive compounds, allow for a human subject with low vision to be exposed to bright light without the negative effect of glare that would be expected with typical vision restoration treatments. Exemplary assessments for glare tolerance are described, e.g., in Fotios et al., LEUKOS (2020) doi: 10.1080/15502724.2020.1803082. In some embodiments, the treating comprises administering the composition comprising a compound of formula (I) into one or both eyes of the subject as described herein.

In some embodiments, the treating comprises reducing non-specific, non-stimulus generated, random electrical activity of the subject's retina, also known as “ringing” of the eye as perceived by the brain. An unexpected advantage of the photoreactive compounds of formula (I) described herein is that they are capable of improving vision in a human subject without causing “ringing” of the eyes. In some embodiments, the treating comprises administering the composition comprising a compound of formula (I) into one or both eyes of the subject as described herein.

In some embodiments, the treating comprises increasing measures of the subject's functional vision. In some embodiments, a subject's functional vision may be measured via a Functional Vision Assessment (FVA). In some embodiments, the FVA comprises testing the subject's visual acuity, visual field, contrast discrimination, sensitivity, color vision, and/or light sensitivity. In some embodiments, the treating comprises increasing measures of the subject's visual quality of life. In some embodiments, a subject's visual quality of life may be assessed via a survey, e.g., the National Eye Institute Visual Functioning Questionnaire 25 (VFQ-25). In some embodiments, the subject's VFQ-25 score increases by about 1 to 5 points, or about 2 to 4 points at about 2 days, about 7 days, about 10 days, about 15 days, or about 30 days after the treatment. In some embodiments, the treatment comprises administering the composition comprising a compound of formula (I) into one eye of the subject as described herein, wherein the increase in the subject's VFQ-25 score is based on results from both eyes of the subject. In some embodiments, the treatment comprises administering the composition comprising a compound of formula (I) into both eyes of the subject as described herein.

### Compounds

In some embodiments, the method provided herein comprises administering to the eye of the subject via intravitreal injection a composition comprising a compound of formula (I):

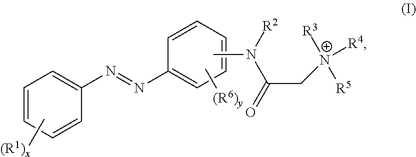

or a pharmaceutically acceptable salt thereof, wherein R¹, R², R³, R⁴, R⁵, R⁶, x, and y are as described herein.

In some embodiments, each R¹ is independently selected from C₁₋₁₀ alkyl, substituted C₁₋₁₀ alkyl, —NR¹⁰R¹¹, —NR¹²C(O)R¹³, C₂₋₁₀ alkenyl, substituted C₂₋₁₀ alkenyl, C₂₋₁₀ alkynyl, substituted C₂₋₁₀ alkynyl, C₆₋₂₀ aryl; substituted C₆₋₂₀ aryl, heteroaryl, heterocyclyl, heterocyclooxy, heterocyclothio, heteroarylamino, heterocycloamino, C₄₋₁₀ cycloalkyl, substituted C₄₋₁₀ cycloalkyl, C₄₋₁₀ cycloalkenyl, substituted C₄₋₁₀ cycloalkenyl, cyano, halo, —OR¹⁰, —C(O)OR¹⁰, and —S(O)₂R¹⁰.

In some embodiments, R³, R⁴, and R⁵ are independently selected from hydrogen, C₂₋₈ alkyl, substituted C₂₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl.

In some embodiments, x is an integer from 0 to 5. In some embodiments, y is an integer from 0 to 4.

In some embodiments, R² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl.

In some embodiments, each R⁶ is independently selected from hydrogen, C₁₋₁₀ alkyl, substituted C₁₋₁₀ alkyl, —NR¹⁰R¹¹, —NR¹²C(O)R¹³, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₋₁₀ cycloalkyl, substituted C₄₋₁₀ cycloalkyl, C₄₋₁₀ cycloalkenyl, substituted C₄₋₁₀ cycloalkenyl, cyano, halo, —OR¹⁰, —C(O)OR¹⁰, —S(O)R¹⁰, and —S(O)₂R¹⁰.

In some embodiments, R¹⁰ and R¹¹ are independently selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl.

In some embodiments, R¹² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl.

In some embodiments, R¹³ is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆-C₁₀ aryl, substituted C₆₋₂₀ aryl, C₄₋₁₀ cycloalkyl, substituted C₄₋₁₀ cycloalkyl, C₄₋₁₀ cycloalkenyl, substituted C₄₋₁₀ cycloalkenyl, —CH₂—N(CH₂CH₃)₃⁺, and —CH₂—SO₃⁻.

In some embodiments, each R¹ is independently selected from halo, C₁₋₁₀ alkyl,

—NR¹⁰R¹¹, and —NR¹²C(O)R¹³.

In some embodiments, R² is hydrogen or ethyl.

In some embodiments, x is 0 or 1. In some embodiments, y is 0 or 1.

In some embodiments, each of R³, R⁴, and R⁵ is ethyl.

In some embodiments, R⁶ is halo or C₁₋₁₀ alkyl.

In some embodiments, R¹⁰ or R¹¹ are independently selected from hydrogen, C₁₋₁₀ alkyl, and substituted C₁₋₁₀ alkyl.

In some embodiments, R¹² is hydrogen.

In some embodiments, R¹³ is selected from C₁₋₁₀ alkyl, substituted C₁₋₁₀ alkyl, C₂₋₈ alkenyl, and C₆₋₁₀ aryl.

Although Formula (I) depicts an azo bond in the trans configuration, it is to be understood that the formula encompasses both the cis and trans configuration, unless otherwise indicated herein.

In some embodiments, the compound of formula (I) is selected from the following structures:

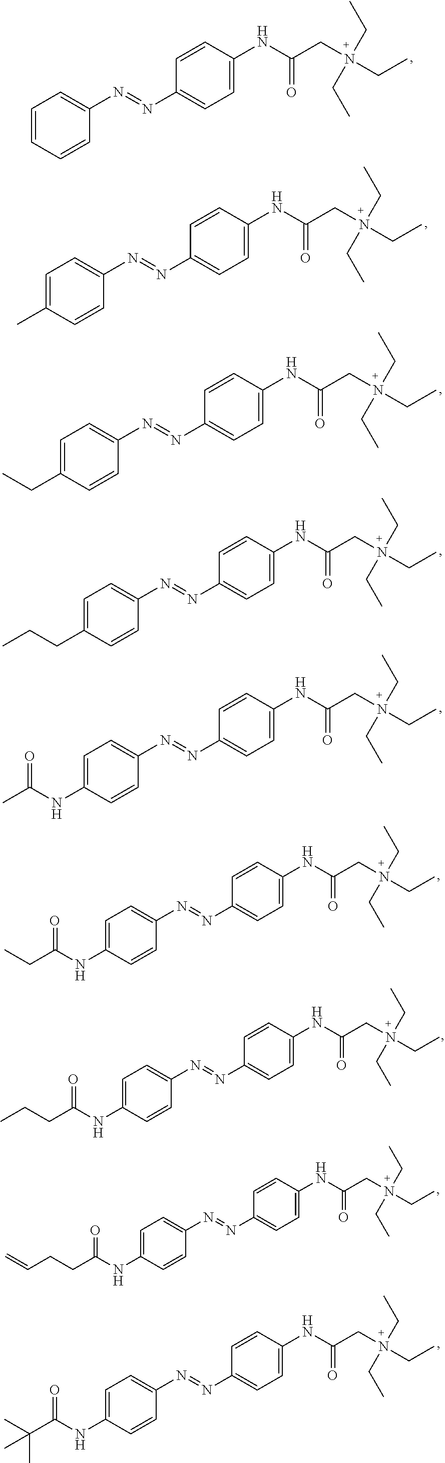

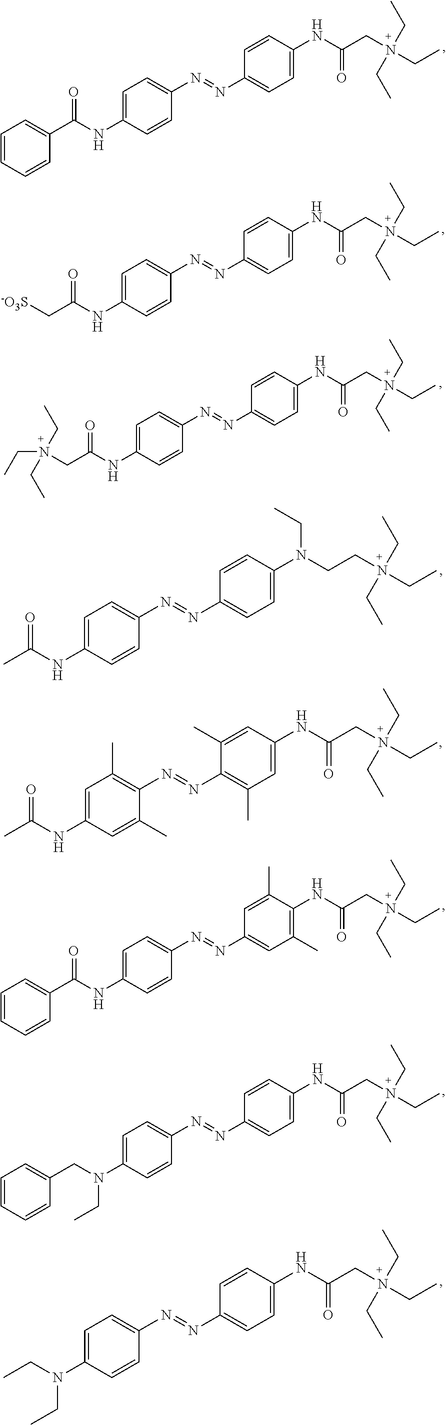

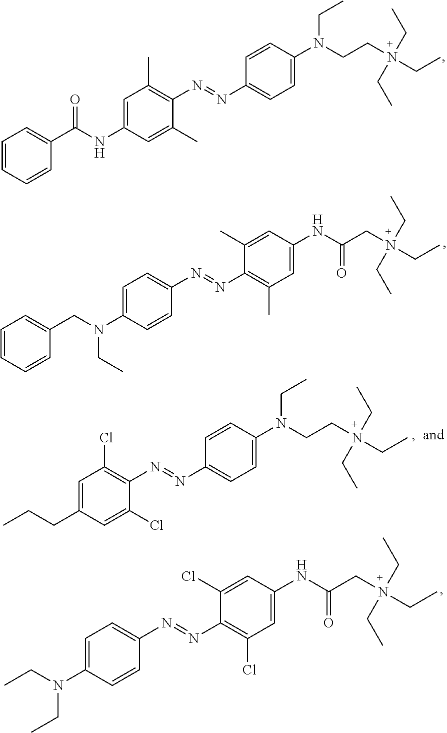

or pharmaceutically acceptable salts thereof, wherein the azo (N═N) bond in the compounds of formula (I) may be either cis or trans, unless otherwise indicated herein.

In some embodiments, the compound of formula (I) has the structure:

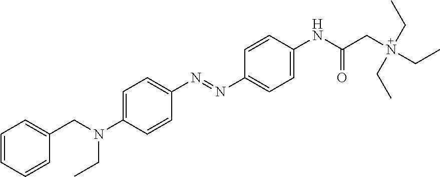

or a pharmaceutically acceptable salt thereof, wherein the azo bond in the structure may be either cis or trans.

The term “pharmaceutically acceptable salt” refers to salts that retain the biological effectiveness and properties of a compound, which are not biologically or otherwise undesirable for use in a pharmaceutical. In some embodiments, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Exemplary salts are described in WO 87/05297.

As used herein, “Cₐ to C_{b}” or “C_{a-b}” in which “a” and “b” are integers refer to the number of carbon atoms in the specified group. That is, the group can contain from “a” to “b,” inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of Group 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group of the compounds may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “haloalkyl” refers to a straight- or branched-chain alkyl group having from 1 to 12 carbon atoms in the chain, substituting one or more hydrogens with halogens. Examples of haloalkyl groups include, but are not limited to, —CF₃, —CHF₂, —CH₂F, —CH₂CF₃, —CH₂CHF₂, —CH₂CH₂F, —CH₂CH₂Cl, —CH₂CF₂CF₃ and other groups that in light of the ordinary skill in the art and the teachings provided herein, would be considered equivalent to any one of the foregoing examples.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. In various embodiments, the heteroalkyl may have from 1 to 4 heteroatoms, from 1 to 3 heteroatoms, 1 or 2 heteroatoms, or 1 heteroatom. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. In various embodiments, a heteroaryl contains from 1 to 4 heteroatoms, from 1 to 3 heteroatoms, from 1 to 2 heteroatoms, or 1 heteroatom. For example, in various embodiments, a heteroaryl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle [2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations.

In various embodiments, a heterocyclyl contains from 1 to 4 heteroatoms, from 1 to 3 heteroatoms, from 1 to 2 heteroatoms, or 1 heteroatom. For example, in various embodiments, a heterocyclyl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-sulfonamido” group refers to a “—N(R_{A})SO₂R_{B}” group in which R_{A} and R_{b} are each independently selected from hydrogen, C₆₋₁₀ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-carbamyl” group refers to a “—OC(═O)NR_{A}R_{B}” group in which R_{A} and R_{B} are each independently selected from hydrogen, C₆₋₁₀ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})OC(═O)R_{B}” group in which R_{A} and R_{B} are each independently selected from hydrogen, C₆₋₁₀ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-thiocarbamyl” group refers to a “—OC(═S)NR_{A}R_{B}” group in which R_{A} and R_{B} are each independently selected from hydrogen, C₆₋₁₀ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-thiocarbamyl” group refers to an “—N(R_{A})OC(═S)R_{B}” group in which R_{A} and R_{B} are each independently selected from hydrogen, C₆₋₁₀ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-amido” group refers to a “—C(═O)NR_{A}R_{B}” group in which R_{A} and R_{B} are each independently selected from hydrogen, C₆₋₁₀ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-amido” group refers to a “—N(R_{A})C(═O)R_{B}” group in which R_{A} and R_{B} are each independently selected from hydrogen, C₆₋₁₀ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, C₆₋₁₀ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substitutents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, quaternary ammonium, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

In some embodiments, substituted group(s) is (are) substituted with one or more substituent(s) individually and independently selected from C₁-C₄ alkyl, amino, hydroxy, and halogen.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

In some embodiments, the compound of formula (I) as described herein is administered in a composition. In some embodiments, the concentration of the compound of formula (I) in the composition is about 0.1 mM to about 10 mM. In some embodiments, the concentration of the compound of formula (I) in the composition is about 0.2 mM to about 5 mM. In some embodiments, the concentration of the compound of formula (I) in the composition is about 0.3 mM to about 2 mM. In some embodiments, the concentration of the compound of formula (I) in the composition is about 0.5 mM to about 1 mM.

### Cyclodextrin

In some embodiments, the composition described herein, comprising a compound of formula (I), further comprises a cyclodextrin. In some embodiments, the cyclodextrin in the composition is a cyclodextrin as described in WO 2022/093652. In some embodiments, the cyclodextrin is an alkylated cyclodextrin. As used herein, an “alkylated cyclodextrin” is a cyclodextrin wherein one or more hydrogen atoms on the hydroxy substituents on the cyclodextrin are replaced with an alkyl group, which may be optionally substituted with other substituents. In one embodiment, alkylated cyclodextrins for use as described herein have the structure of Formula (II):

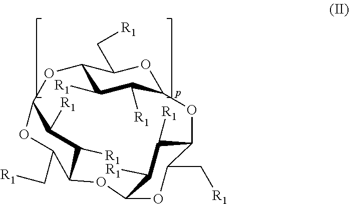

or pharmaceutically acceptable salts thereof, wherein p is 4, 5, or 6, and R₁ is independently selected at each occurrence from —OH and optionally substituted —O—C₁-C₈ alkyl, wherein at least one R₁ is an optionally substituted alkyl.

Optional substituents for substituting —O—C₁-C₈ alkyl include C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O).

In some embodiments, p is 5 (i.e., the cyclodextrin is a β-cyclodextrin). In some embodiments, the alkylated cyclodextrin is a sulfoalkylether-β-cyclodextrin. For example, in some embodiments, at least one R₁ is O—(C₂-C₆ alkylene)-SO₃⁻-T, wherein T is independently selected at each occurrence from pharmaceutically acceptable cations. Suitable examples of T include H⁺, alkali metals (e.g., Li⁺, Na⁺, K⁺), alkaline earth metals (e.g., Ca⁺², Mg⁺²), ammonium ions and amine cations such as the cations of (C₁-C₆)-alkylamines, piperidine, pyrazine, (C₁-C₆)-alkanolamine, ethylenediamine and (C₄-C₈)-cycloalkanolamine among others, and combinations thereof.

In some embodiments, the alkylated cyclodextrin has the structure of Formula (III):

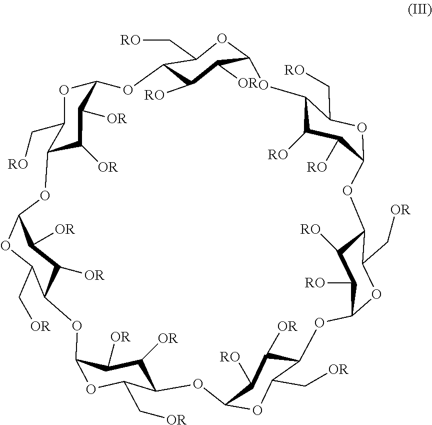

wherein each R is independently —H or —(CH₂)₄—SO₃—Na⁺, and the average degree of substitution with —(CH₂)₄—SO₃—Na⁺ of all cyclodextrin molecules in the composition is from 6 to 7.1. For example, in some embodiments, the alkylated cyclodextrin may be CAPTISOL®.

In the compositions described herein, individual molecules of cyclodextrin in the composition may have varying degrees of substitution for a specified substituent. Accordingly, it is common to characterize such compositions by an average degree of substitution (ADS) for a specified substituent. Thus, for example, an ADS of 6 to 7.1 indicates that, although each molecule of cyclodextrin in the composition has an integer degree of substitution for a specified substituent, there is a distribution of such degrees of substitution in the composition, resulting in an average of 6 to 7.1.

Further exemplary sulfoalkyl ether (SAE)-CD derivatives include:

TABLE 1

| SAEₓ-α-CD | SAEₓ-β-CD | SAEₓ-γ-CD |
|---|---|---|
| (Sulfoethyl ether)ₓ-α-CD | (Sulfoethyl ether)ₓ-β-CD | (Sulfoethyl ether)ₓ-γ-CD |
| (Sulfopropyl ether)ₓ-α-CD | (Sulfopropyl ether)ₓ-β-CD | (Sulfopropyl ether)ₓ-γ-CD |
| (Sulfobutyl ether)ₓ-α-CD | (Sulfobutyl ether)ₓ-β-CD | (Sulfobutyl ether)ₓ-γ-CD |
| (Sulfopentyl ether)ₓ-α-CD | (Sulfopentyl ether)ₓ-β-CD | (Sulfopentyl ether)ₓ-γ-CD |
| (Sulfohexyl ether)ₓ-α-CD | (Sulfohexyl ether)ₓ-β-CD | (Sulfohexyl ether)ₓ-γ-CD |

wherein x denotes the average degree of substitution. In some embodiments, the alkylated cyclodextrins are formed as salts.

Various embodiments of a sulfoalkyl ether cyclodextrin include eicosa-O-(methyl)-6G-O-(4-sulfobutyl)-β-cyclodextrin, heptakis-O-(sulfomethyl)-tetradecakis-O-(3-sulfopropyl)-β-cyclodextrin, heptakis-O-[(1,1-dimethylethyl)dimethylsilyl]-tetradecakis-O-(3-sulfopropyl)-β-cyclodextrin, heptakis-O-(sulfomethyl)-tetradecakis-O-(3-sulfopropyl)-β-cyclodextrin, and heptakis-O-[(1,1-dimethylethyl)dimethylsilyl]-tetradecakis-O-(sulfomethyl)-β-cyclodextrin. Other known alkylated cyclodextrins containing a sulfoalkyl moiety include sulfoalkylthio and sulfoalkylthioalkyl ether derivatives such as octakis-(S-sulfopropyl)-octathio-γ-cyclodextrin, octakis-O-[3-[(2-sulfoethyl)thio]propyl]-β-cyclodextrin], and octakis-S-(2-sulfoethyl)-octathio-γ-cyclodextrin.

In some embodiments, an alkylated cyclodextrin composition of the present disclosure is a sulfoalkyl ether-β-cyclodextrin composition having an ADS of 2 to 9, 4 to 8, 4 to 7.5, 4 to 7, 4 to 6.5, 4.5 to 8, 4.5 to 7.5, 4.5 to 7, 5 to 8, 5 to 7.5, 5 to 7, 5.5 to 8, 5.5 to 7.5, 5.5 to 7, 5.5 to 6.5, 6 to 8, 6 to 7.5, 6 to 7.1, 6.5 to 7.1, 6.2 to 6.9, or 6.5 per alkylated cyclodextrin, and the remaining substituents are —H.

In some embodiments, R¹ of Formula (II) is —OH or unsubstituted —O—C₁-C₈ alkyl. Such alkylated cyclodextrins are known as alkylether (AE)-CDs. Exemplary AE-CD derivatives include:

TABLE 2

| (Alkylether)_{y}-α-CD | (Alkylether)_{y}-β-CD | (Alkylether)_{y}-γ-CD |
|---|---|---|
| ME_{y}-α-CD | ME_{y}-β-CD | ME_{y}-γ-CD |
| EE_{y}-α-CD | EE_{y}-β-CD | EE_{y}-γ-CD |
| PE_{y}-α-CD | PE_{y}-β-CD | PE_{y}-γ-CD |
| BE_{y}-α-CD | BE_{y}-β-CD | BE_{y}-γ-CD |
| PtE_{y}-α-CD | PtE_{y}-β-CD | PtE_{y}-γ-CD |
| HE_{y}-α-CD | HE_{y}-β-CD | HE_{y}-γ-CD |

wherein ME denotes methyl ether, EE denotes ethyl ether, PE denotes propyl ether, BE denotes butyl ether, PtE denotes pentyl ethyl, HE denotes hexyl ether, and y denotes the average degree of substitution.

In some embodiments, at least one R₁ of Formula (II) is —O—C₁-C₆ alkyl substituted with hydroxyl (e.g., hydroxypropyl-β-cyclodextrin). Further exemplary hydroxyalkyl ether (HAE)-CD derivatives include:

TABLE 3

| (HAE)_{z}-α-CD | (HAE)_{z}-β-CD | (HAE)_{z}-γ-CD |
|---|---|---|
| HMEz-α-CD | HMEz-β-CD | HMEz-γ-CD |
| HEEz-α-CD | HEEz-β-CD | HEEz-γ-CD |
| HPEz-α-CD | HPEz-β-CD | HPEz-γ-CD |
| HBEz-α-CD | HBEz-β-CD | HBEz-γ-CD |
| HPtEz-α-CD | HPtEz-β-CD | HPtEz-γ-CD |
| HHEz-α-CD | HHEz-β-CD | HHEz-γ-CD |

wherein HME denotes hydroxymethyl ether, HEE denotes hydroxyethyl ether, HPE denotes hydroxypropyl ether, HBE denotes hydroxybutyl ether, HPtE denotes hydroxypentyl ether, HHE denotes hydroxyhexyl ether, and z denotes the average degree of substitution.

In some embodiments, alkylated cyclodextrins are provided having mixed substituents (e.g., including both sulfoalkylether and alkylether substituents (SAE-AE-CDs)). Specific embodiments of the such derivatives of include those wherein: 1) the alkylene moiety of the SAE has the same number of carbons as the alkyl moiety of the AE; 2) the alkylene moiety of the SAE has a different number of carbons than the alkyl moiety of the AE; 3) the alkyl and alkylene moieties are independently selected from the group consisting of a straight chain or branched moiety; 4) the alkyl and alkylene moieties are independently selected from the group consisting of a saturated or unsaturated moiety; 5) the ADS for the SAE group is greater than or approximates the ADS for the AE group; or 6) the ADS for the SAE group is less than the ADS for the AE group. Some embodiments include a SAE-HAE-CD.

The alkylated cyclodextrin can include SAE-CD, HAE-CD, SAE-HAE-CD, HANE-CD, HAE-AE-CD, HAE-SAE-CD, AE-CD, SAE-AE-CD, neutral cyclodextrin, anionic cyclodextrin, cationic cyclodextrin, halo-derivatized cyclodextrin, amino-derivatized cyclodextrin, nitrile-derivatized cyclodextrin, aldehyde-derivatized cyclodextrin, carboxylate-derivatized cyclodextrin, sulfate-derivatized cyclodextrin, sulfonate-derivatized cyclodextrin, mercapto-derivatized cyclodextrin, alkylamino-derivatized cyclodextrin, or succinyl-derivatized cyclodextrin.

In some embodiments, alkylated cyclodextrins such as mixed ether alkylated cyclodextrins include, by way of example, those listed Table 4 below:

TABLE 4

| Mixed ether | Mixed ether | Mixed ether |
|---|---|---|
| CD derivative | CD derivative | CD derivative |
| Sulfobutyl-hydroxybutyl-CD | Sulfopropyl-hydroxybutyl-CD | Sulfoethyl-hydroxybutyl-CD |
| (SBE-HBE-CD) | (SPE-HBE-CD) | (SEE-HBE-CD) |
| Sulfobutyl-hydroxypropyl-CD | Sulfopropyl-hydroxypropyl-CD | Sulfoethyl-hydroxypropyl-CD |
| (SBE-HPE-CD) | (SPE-HPE-CD) | (SEE-HPE-CD) |
| Sulfobutyl-hydroxyethyl-CD | Sulfopropyl-hydroxyethyl-CD | Sulfoethyl-hydroxyethyl-CD |
| (SBE-HEE-CD) | (SPE-HEE-CD) | (SEE-HEE-CD) |
| Sulfobutyl-hydroxybutenyl-CD | Sulfopropyl-hydroxybutenyl-CD | Sulfoethyl-hydroxybutenyl-CD |
| (SBE-HBNE-CD) | (SPE-HBNE-CD) | (SEE-HBNE-CD) |
| Sulfobutyl-ethyl | Sulfopropyl-ethyl | Sulfoethyl-ethyl |
| (SBE-EE-CD) | (SPE-EE-CD) | (SEE-EE-CD) |
| Sulfobutyl-methyl | Sulfopropyl-methyl | Sulfoethyl-methyl |
| (SBE-ME-CD) | (SPE-ME-CD) | (SEE-ME-CD) |
| Sulfobutyl-propyl | Sulfopropyl-propyl | Sulfoethyl-propyl |
| (SBE-PE-CD) | (SPE-PE-CD) | (SEE-PE-CD) |
| Sulfobutyl-butyl | Sulfopropyl-butyl | Sulfoethyl-butyl |
| (SBE-BE-CD) | (SPE-BE-CD) | (SEE-BE-CD) |
| Sulfobutyl-carboxymethyl-CD | Sulfopropyl-carboxymethyl-CD | Sulfoethyl-carboxymethyl-CD |
| (SBE-CME-CD) | (SPE-CME-CD) | (SEE-CME-CD) |
| Sulfobutyl-carboxyethyl-CD | Sulfopropyl-carboxyethyl-CD | Sulfoethyl-carboxyethyl-CD |
| (SBE-CEE-CD) | (SPE-CEE-CD) | (SEE-CEE-CD) |
| Sulfobutyl-acetate-CD | Sulfopropyl-acetate-CD | Sulfoethyl-acetate-CD |
| (SBE-AA-CD) | (SPE-AA-CD) | (SEE-AA-CD) |
| Sulfobutyl-propionate-CD | Sulfopropyl-propionate-CD | Sulfoethyl-propionate-CD |
| (SBE-PA-CD) | (SPE-PA-CD) | (SEE-PA-CD) |
| Sulfobutyl-butyrate-CD | Sulfopropyl-butyrate-CD | Sulfoethyl-butyrate-CD |
| (SBE-BA-CD) | (SPE-BA-CD) | (SEE-BA-CD) |
| Sulfobutyl-methoxycarbonyl-CD | Sulfopropyl-methoxycarbonyl-CD | Sulfoethyl-methoxycarbonyl-CD |
| (SBE-MC-CD) | (SPE-MC-CD) | (SEE-MC-CD) |
| Sulfobutyl-ethoxycarbonyl-CD | Sulfopropyl-ethoxycarbonyl-CD | Sulfoethyl-ethoxycarbonyl-CD |
| (SBE-EC-CD) | (SPE-EC-CD) | (SEE-EC-CD) |
| Sulfobutyl-propoxycarbonyl-CD | Sulfopropyl-propoxycarbonyl-CD | Sulfoethyl-propoxycarbonyl-CD |
| (SBE-PC-CD) | (SPE-PC-CD) | (SEE-PC-CD) |
| Hydroxybutyl-hydroxybutenyl-CD | Hydroxypropyl-hydroxybutenyl-CD | Hydroxyethyl-hydroxybutenyl-CD |
| (HBE-HBNE-CD) | (HPE-HBNE-CD) | (HEE-HBNE-CD) |
| Hydroxybutyl-ethyl-CD | Hydroxypropyl-ethyl-CD | Hydroxyethyl-ethyl-CD |
| (HBE-EE-CD) | (HPE-EE-CD) | (HEE-EE-CD) |
| Hydroxybutyl-methyl-CD | Hydroxypropyl-methyl-CD | Hydroxyethyl-methyl-CD |
| (HBE-ME-CD) | (HPE-ME-CD) | (HEE-ME-CD) |
| Hydroxybutyl-propyl-CD | Hydroxypropyl-propyl-CD | Hydroxyethyl-propyl-Cd |
| (HBE-PE-CD) | (HPE-PE-CD) | (HEE-PE-CD) |
| Hydroxybutyl-butyl | Hydroxypropyl-butyl | Hydroxyethyl-butyl |
| (HBE-BE-CD) | (HPE-BE-CD) | (HEE-BE-CD) |
| Hydroxybutyl-carboxymethyl-CD | Hydroxypropyl-carboxymethyl-CD | Hydroxyethyl-carboxymethyl-CD |
| (HBE-CME-CD) | (HPE-CME-CD) | (HEE-CME-CD) |
| Hydroxybutyl-carboxyethyl-CD | Hydroxypropyl-carboxyethyl-CD | Hydroxyethyl-carboxyethyl-CD |
| (HBE-CEE-CD) | (HPE-CEE-CD) | (HEE-CEE-CD) |
| Hydroxybutyl-acetate-CD | Hydroxypropyl-acetate-CD | Hydroxyethyl-acetate-CD |
| (HBE-AA-CD) | (HPE-AA-CD) | (HEE-AA-CD) |
| Hydroxybutyl-propionate-CD | Hydroxypropyl-propionate-CD | Hydroxyethyl-propionate-CD |
| (HBE-PA-CD) | (HPE-PA-CD) | (HEE-PA-CD) |
| Hydroxybutyl-butyrate-CD | Hydroxypropyl-butyrate-CD | Hydroxyethyl-butyrate-CD |
| (HBE-BA-CD) | (HPE-BA-CD) | (HEE-BA-CD) |
| Hydroxybutyl-methoxycarbonyl-CD | Hydroxypropyl-methoxycarbonyl-CD | Hydroxyethyl-methoxycarbonyl-CD |
| (HBE-MC-CD) | (HPE-MC-CD) | (HEE-MC-CD) |
| Hydroxybutyl-ethoxycarbonyl-CD | Hydroxypropyl-ethoxycarbonyl-CD | Hydroxyethyl-ethoxycarbonyl-CD |
| (HBE-EC-CD) | (HPE-EC-CD) | (HEE-EC-CD) |
| Hydroxybutyl-propoxycarbonyl-CD | Hydroxypropyl-propoxycarbonyl-CD | Hydroxyethyl-propoxycarbonyl-CD |
| (HBE-PC-CD) | (HPE-PC-CD) | (HEE-PC-CD) |
| Hydroxybutenyl-ethyl-CD | Hydroxypropenyl-ethyl-CD | Hydroxypentenyl-ethyl-CD |
| (HBNE-EE-CD) | (HPNE-EE-CD) | (HPTNE-EE-CD) |
| Hydroxybutenyl-methyl-CD | Hydroxypropenyl-methyl-CD | Hydroxypentenyl-methyl-CD |
| (HBNE-ME-CD) | (HPNE-ME-CD) | (HPTNE-ME-CD) |
| Hydroxybutenyl-propyl-CD | Hydroxypropenyl-propyl-CD | Hydroxypentenyl-propyl-CD |
| (HBNE-PE-CD) | (HPNE-PE-CD) | (HPTNE-PE-CD) |
| Hydroxybutenyl-butyl-CD | Hydroxypropenyl-butyl-CD | Hydroxypentenyl-butyl-CD |
| (HBNE-BE-CD) | (HPNE-BE-CD) | (HPTNE-BE-CD) |
| Hydroxybutenyl-carboxymethyl-CD | Hydroxypropenyl-carboxymethyl-CD | Hydroxypentenyl-carboxymethyl-CD |
| (HBNE-CME-CD) | (HPNE-CME-CD) | (HPTNE-CME-CD) |
| Hydroxybutenyl-carboxyethyl-CD | Hydroxypropenyl-carboxyethyl-CD | Hydroxypentenyl-carboxyethyl-CD |
| (HBNE-CEE-CD)- | (HPNE-CEE-CD) | (HPTNE-CEE-CD) |
| Hydroxybutenyl-acetate-CD | Hydroxypropenyl-acetate-CD | Hydroxypentenyl-acetate-CD |
| (HBNE-AA-CD) | (HPNE-AA-CD) | (HPTNE-AA-CD) |
| Hydroxybutenyl-propionate-CD | Hydroxypropenyl-propionate-CD | Hydroxypentenyl-propionate-CD |
| (HBNE-PA-CD) | (HPNE-PA-CD) | (HPTNE-PA-CD) |
| Hydroxybutenyl-butyrate-CD | Hydroxypropenyl-butyrate-CD | Hydroxypentenyl-butyrate-CD |
| (HBNE-BA-CD) | (HPNE-BA-CD) | (HPTNE-BA-CD) |
| Hydroxybutenyl-methoxycarbonyl-CD | Hydroxypropenyl-methoxycarbonyl-CD | Hydroxypentenyl-methoxycarbonyl-CD |
| (HBNE-MC-CD) | (HPNE-MC-CD) | (HPTNE-MC-CD) |
| Hydroxybutenyl-ethoxycarbonyl-CD | Hydroxypropenyl-ethoxycarbonyl-CD | Hydroxypentenyl-ethoxycarbonyl-CD |
| (HBNE-EC-CD) | (HPNE-EC-CD) | (HPTNE-EC-CD) |
| Hydroxybutenyl-propoxycarbonyl-CD | Hydroxypropenyl-propoxycarbonyl-CD | Hydroxypentenyl-propoxycarbonyl-CD |
| (HBNE-PC-CD) | (HPNE-PC-CD) | (HPTNE-PC-CD) |

Additional examples of alkylated cyclodextrins that may be included in the compositions described herein are described in U.S. Pat. Nos. 5,438,133, 6,479,467, and 6,610,671, the disclosure of each of which is incorporated by reference herein in its entirety.

Within a given alkylated cyclodextrin composition, the substituents of the alkylated cyclodextrin(s) thereof can be the same or different. For example, SAE or HAE moieties can have the same type or different type of alkylene (alkyl) radical upon each occurrence in an alkylated cyclodextrin composition. In such embodiments, the alkylene radical in the SAE or HAE moiety can be ethyl, propyl, butyl, pentyl or hexyl in each occurrence in an alkylated cyclodextrin composition.

The alkylated cyclodextrins can differ in their degree of substitution by functional groups, the number of carbons in the functional groups, their molecular weight, the number of glucopyranose units contained in the base cyclodextrin used to form the derivatized cyclodextrin and or their substitution patterns. In addition, the derivatization of a cyclodextrin with functional groups occurs in a controlled, although not exact manner. For this reason, the degree of substitution is actually a number representing the average number of functional groups per cyclodextrin (for example, SBE₇-β-CD has an average of 7 substitutions per cyclodextrin). Thus, it has an average degree of substitution (“ADS”) of 7. In some embodiments, the ADS may be determined by techniques including capillary electrophoresis (CE), high performance liquid chromatography (HPLC), nuclear magnetic resonance (NMR) spectroscopy, or a combination thereof. In addition, the regiochemistry of substitution of the hydroxyl groups of the cyclodextrin is variable with regard to the substitution of specific hydroxyl groups of the hexose ring. For this reason, substitution of the different hydroxyl groups is likely to occur during manufacture of the derivatized cyclodextrin, and a particular derivatized cyclodextrin will possess a preferential, although not exclusive or specific, substitution pattern. Given the above, the molecular weight of a particular derivatized cyclodextrin composition can vary from batch to batch.

In a single parent cyclodextrin molecule, there are 3v+6 hydroxyl moieties available for derivatization. Where v=4 (α-cyclodextrin), “y” the degree of substitution for the moiety can range in value from 1 to 18. Where v=5 (β-cyclodextrin), “y” the degree of substitution for the moiety can range in value from 1 to 21. Where v=6 (γ-cyclodextrin), “y” the degree of substitution for the moiety can range in value from 1 to 24. In general, “y” also ranges in value from 1 to 3v+g, where g ranges in value from 0 to 5. In some embodiments, “y” ranges from 1 to 2v+g, or from 1 to 1v+g.

The degree of substitution (“DS”) for a specific moiety (SAE, HAE or AE, for example) is a measure of the number of SAE (HAE or AE) substituents attached to an individual cyclodextrin molecule, in other words, the moles of substituent per mole of cyclodextrin. Therefore, each substituent has its own DS for an individual alkylated cyclodextrin species. The average degree of substitution (“ADS”) for a substituent is a measure of the total number of substituents present per cyclodextrin molecule for the distribution of alkylated cyclodextrins within an alkylated cyclodextrin composition of the present disclosure. Therefore, SAE₄-CD has an ADS (per CD molecule) of 4.

Some embodiments of the present disclosure include those wherein: 1) more than half of the hydroxyl moieties of the alkylated cyclodextrin are derivatized; 2) half or less than half of the hydroxyl moieties of the alkylated cyclodextrin are derivatized; 3) the substituents of the alkylated cyclodextrin are the same upon each occurrence; 4) the substituents of the alkylated cyclodextrin comprise at least two different substituents; or 5) the substituents of the alkylated cyclodextrin comprise one or more of substituents selected from the group consisting of unsubstituted alkyl, substituted alkyl, halide (halo), haloalkyl, amine (amino), aminoalkyl, aldehyde, carbonylalkyl, nitrile, cyanoalkyl, sulfoalkyl, hydroxyalkyl, carboxyalkyl, thioalkyl, unsubstituted alkylene, substituted alkylene, aryl, arylalkyl, heteroaryl, and heteroarylalkyl.

Alkylated cyclodextrin compositions can comprise multiple alkylated cyclodextrin molecules differing in degree of substitution. For example, an alkylated cyclodextrin molecule can have 1, 2, 3, 4, 5, 6, 7, 8, 9, 10, 11, 12, or more of the hydroxyl groups of the parent cyclodextrin functionalized with a substituent, e.g., a sulfoalkyl ether. In such compositions, the average degree of substitution (ADS) can be calculated, as described herein, based on the relative amounts of alkylated cyclodextrin molecules having a particular degree of substitution. As a consequence, the ADS for SAE of a SAE-CD derivative composition represents a weighted average of the degree of substitution of the individual SAE-CD molecules in the composition. For example, a SAE_{5.2}-CD composition comprises a distribution of multiple SAEx-CD molecules, wherein “x” (the DS for SAE groups) can range from integers having values of 1 to 12 for individual cyclodextrin molecules; however, the population of SAE-cyclodextrin molecules is such that the average value for “x” (the ADS for SAE groups) is 5.2.

The alkylated cyclodextrin compositions can have a high to moderate to low ADS. The alkylated cyclodextrin compositions can also have a wide or narrow “span,” which is the number of alkylated cyclodextrin molecules with differing degrees of substitution within an alkylated cyclodextrin composition. For example, an alkylated cyclodextrin composition comprising a single species of alkylated cyclodextrin having a single degree of substitution is said to have a span of one, and in such a case, the degree of substitution for the alkylated cyclodextrin molecule would equal the ADS of its alkylated cyclodextrin composition. An electropherogram, for example, of an alkylated cyclodextrin with a span of one should have only one alkylated cyclodextrin species with respect to degree of substitution. An alkylated cyclodextrin composition having a span of two comprises two individual alkylated cyclodextrin species differing in their degree of substitution, and its electropherogram, for example, would indicate two different alkylated cyclodextrin species differing in degree of substitution. Likewise, the span of an alkylated cyclodextrin composition having a span of three comprises three individual alkylated cyclodextrin species differing in their degree of substitution. The span of an alkylated cyclodextrin composition typically ranges from 5 to 15, or 7 to 12, or 8 to 11.

A parent cyclodextrin includes a secondary hydroxyl group on the C-2 and C-3 positions of the glucopyranose residues forming the cyclodextrin and a primary hydroxyl on the C-6 position of the same. Each of these hydroxyl moieties is available for derivatization by substituent precursor. Depending upon the synthetic methodology employed, the substituent moieties can be distributed randomly or in a somewhat ordered manner among the available hydroxyl positions. The regioisomerism of derivatization by the substituent can also be varied as desired. The regioisomerism of each composition is independently selected. For example, a majority of the substituents present can be located at a primary hydroxyl group or at one or both of the secondary hydroxyl groups of the parent cyclodextrin. In some embodiments, the primary distribution of substituents is C-3>C-2>C-6, while in other embodiments the primary distribution of substituents is C-2>C-3>C-6. Some embodiments of the present disclosure include an alkylated cyclodextrin molecule wherein a minority of the substituent moieties is located at the C-6 position, and a majority of the substituent moieties is located at the C-2 and/or C-3 position. Still other embodiments of the present disclosure include an alkylated cyclodextrin molecule wherein the substituent moieties are substantially evenly distributed among the C-2, C-3, and C-6 positions.

An alkylated cyclodextrin composition comprises a distribution of plural individual alkylated cyclodextrin species, each species having an individual degree of substitution (“IDS”). The content of each of the cyclodextrin species in a particular composition can be quantified using capillary electrophoresis. The method of analysis (capillary electrophoresis, for example, for charged alkylated cyclodextrins) is sufficiently sensitive to distinguish between compositions having only 5% of one alkylated cyclodextrin and 95% of another alkylated cyclodextrin from starting alkylated cyclodextrin compositions containing a single alkylated cyclodextrin.

The above-mentioned variations among the individual species of alkylated cyclodextrins in a distribution can lead to changes in the complexation equilibrium constant K_{1:1} which in turn will affect the required molar ratios of the derivatized cyclodextrin to active agent. The equilibrium constant is also somewhat variable with temperature and allowances in the ratio are required such that the agent remains solubilized during the temperature fluctuations that can occur during manufacture, storage, transport, and use. The equilibrium constant can also vary with pH and allowances in the ratio can be required such that the agent remains solubilized during pH fluctuations that can occur during manufacture, storage, transport, and use. The equilibrium constant can also vary due to the presence of other excipients (e.g., buffers, preservatives, antioxidants). Accordingly, the ratio of derivatized cyclodextrin to active agent can be varied from the ratios set forth herein in order to compensate for the above-mentioned variables.

In some embodiments, the compositions described herein comprise a compound of formula (I) and cyclodextrin, e.g., an alkylated cyclodextrin, in a pre-determined molar ratio. In various embodiments, the molar ratio of cyclodextrin to compound of Formula (I) has a lower limit of 1:1, 2:1, 3:1, 4:1, 5:1, 6:1, 7:1, 8:1, 9:1, 10:1, 15:1, 20:1, 30:1, 50:1, or 100:1 and an upper limit of 10:1, 15:1, 20:1, 30:1, 40:1, 50:1, 70:1, 100:1, 120:1, 150:1, 200:1, 250:1, 300:1, 350:1, 400:1, 450:1, 500:1, 600:1, 750:1, and 1000:1. For example, in various embodiments, the molar ratio of cyclodextrin to compound of Formula (I) is from 1:1 to 500:1, 1:1 to 300:1, 1:1 to 150:1, 1:1 to 100:1, 2:1 to 350:1, 2:1 to 200:1, 2:1 to 100:1, 3:1 to 200:1, 3:1 to 150:1, 3:1, to 100:1, 3:1 to 50:1, 4:1 to 150:1, and 4:1 to 100:1. In some embodiments, the molar ratio of cyclodextrin to the compound of formula (I) is about 20:1 to about 1:1, or about 18:1 to about 2:1, or about 15:1 to about 3:1, or about 10:1 to about 4:1, or about 8:1 to about 5:1. In some embodiments, the molar ratio of cyclodextrin to the compound of formula (I) is about 20:1. In some embodiments, the molar ratio of cyclodextrin to the compound of formula (I) is about 18:1. In some embodiments, the molar ratio of cyclodextrin to the compound of formula (I) is about 15:1. In some embodiments, the molar ratio of cyclodextrin to the compound of formula (I) is about 12:1. In some embodiments, the molar ratio of cyclodextrin to the compound of formula (I) is about 10:1. In some embodiments, the molar ratio of cyclodextrin to the compound of formula (I) is about 8:1. In some embodiments, the molar ratio of cyclodextrin to the compound of formula (I) is about 5:1. In some embodiments, the molar ratio of cyclodextrin to the compound of formula (I) is about 4:1. In some embodiments, the molar ratio of cyclodextrin to the compound of formula (I) is about 3:1. In some embodiments, the molar ratio of cyclodextrin to the compound of formula (I) is about 2:1. In some embodiments, the molar ratio of cyclodextrin to the compound of formula (I) is about 1:1.

Various amounts of the compound of formula (I) can be administered to a single eye in a single administration. In some embodiments, the human subject is administered about 7.5 μg to about 50 μg of the compound of formula (I) to a single eye in a single administration. In some embodiments, the human subject is administered about 5.0 μg to about 10 μg, or about 7.5 μg of the compound of formula (I) to a single eye in a single administration. In some embodiments, about 20 μg to about 30 μg, or about 25 μg of the compound of formula (I) to a single eye in a single administration. In some embodiments, about 40 μg to about 60 μg, or about 50 μg of the compound of formula (I) to a single eye in a single administration. In some embodiments, about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, or about 100 μg of the compound of formula (I) to a single eye in a single administration. In some embodiments, less than 5 μg of the compound of formula (I) to a single eye in a single administration. In some embodiments, greater than 100 μg of the compound of formula (I) to a single eye in a single administration.

### Compositions

The composition provided herein, comprising the compound of formula (I) and, in some embodiments, a cyclodextrin, may be provided as a solid or a liquid formulation. For example, a solid formulation may be provided that is suitable for reconstitution using a diluent prior to administration to a subject. Suitable diluents for reconstitution can include, for example, sterile water or saline solution. When provided as a liquid formulation, or upon reconstitution of a solid formulation, the composition may be an aqueous solution or suspension.

A liquid formulation of the disclosure can be converted to a solid formulation for reconstitution. A reconstitutable solid composition according to the disclosure comprises an active agent (e.g., compound of formula (I)), a derivatized cyclodextrin and optionally at least one other pharmaceutical excipient. A reconstitutable composition can be reconstituted with an aqueous liquid to form a liquid formulation that is preserved. The composition can comprise an admixture (minimal to no presence of an inclusion complex) of a solid derivatized cyclodextrin and an active agent-containing solid and optionally at least one solid pharmaceutical excipient, such that a major portion of the active agent is not complexed with the derivatized cyclodextrin prior to reconstitution. Alternatively, the composition can comprise a solid mixture of a derivatized cyclodextrin and an active agent, wherein a major portion of the active agent is complexed with the derivatized cyclodextrin prior to reconstitution. A reconstitutable solid composition can also comprise a derivatized cyclodextrin and an active agent where substantially all or at least a major portion of the active agent is complexed with the derivatized cyclodextrin.

A reconstitutable solid composition can be prepared according to any of the following processes. A liquid formulation of the disclosure is first prepared, then a solid is formed by lyophilization (freeze-drying), spray-drying, spray freeze-drying, antisolvent precipitation, aseptic spray drying, various processes utilizing supercritical or near supercritical fluids, or other methods known to those of ordinary skill in the art to make a solid for reconstitution.

A liquid vehicle included in a formulation of the disclosure can comprise an aqueous liquid carrier (e.g., water), an aqueous alcohol, an aqueous organic solvent, a non-aqueous liquid carrier, and combinations thereof.

The composition of the present disclosure can include one or more pharmaceutical excipients such as a conventional preservative, antifoaming agent, antioxidant, buffering agent, acidifying agent, alkalizing agent, complexation-enhancing agent, cryoprotectant, electrolyte, glucose, emulsifying agent, oil, plasticizer, solubility-enhancing agent, stabilizer, tonicity modifier, diluent, complexing agents, other excipients known by those of ordinary skill in the art for use in formulations, and combinations thereof.

As used herein, the term “alkalizing agent” is intended to mean a compound used to provide alkaline medium for product stability. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, diethanolamine, organic amine base, alkaline amino acids and trolamine and others known to those of ordinary skill in the art

As used herein, the term “acidifying agent” is intended to mean a compound used to provide an acidic medium for product stability. Such compounds include, by way of example and without limitation, acetic acid, acidic amino acids, citric acid, fumaric acid and other α-hydroxy acids, hydrochloric acid, ascorbic acid, phosphoric acid, sulfuric acid, tartaric acid and nitric acid and others known to those of ordinary skill in the art.

As used herein, a conventional preservative is a compound used to at least reduce the rate at which bioburden increases, but maintains bioburden steady or reduces bioburden after contamination. Such compounds include, by way of example and without limitation, benzalkonium chloride, benzethonium chloride, benzoic acid, benzyl alcohol, cetylpyridinium chloride, chlorobutanol, phenol, phenylethyl alcohol, phenylmercuric nitrate, phenylmercuric acetate, thimerosal, metacresol, myristylgamma picolinium chloride, potassium benzoate, potassium sorbate, sodium benzoate, sodium propionate, sorbic acid, thymol, and methyl, ethyl, propyl or butyl parabens and others known to those of ordinary skill in the art.

As used herein, the term “antioxidant” is intended to mean an agent that inhibits oxidation and thus is used to prevent the deterioration of preparations by the oxidative process. Such compounds include, by way of example and without limitation, acetone, potassium metabisulfite, potassium sulfite, ascorbic acid, ascorbyl palmitate, citric acid, butylated hydroxyanisole, butylated hydroxytoluene, hypophophorous acid, monothioglycerol, propyl gallate, sodium ascorbate, sodium citrate, sodium sulfide, sodium sulfite, sodium bisulfite, sodium formaldehyde sulfoxylate, thioglycolic acid, EDTA, pentetate, and sodium metabisulfite and others known to those of ordinary skill in the art.

As used herein, the term “buffering agent” is intended to mean a compound used to resist change in pH upon dilution or addition of acid or alkali. Such compounds include, by way of example and without limitation, acetic acid, sodium acetate, adipic acid, benzoic acid, sodium benzoate, boric acid, sodium borate, citric acid, glycine, maleic acid, monobasic sodium phosphate, dibasic sodium phosphate, 4-(2-hydroxyethyl)-1-piperazineethanesulfonic acid, lactic acid, tartaric acid, potassium metaphosphate, potassium phosphate, monobasic sodium acetate, sodium bicarbonate, tris, sodium tartrate and sodium citrate anhydrous and dihydrate and others known to those of ordinary skill in the art.

A complexation-enhancing agent can be added to a composition of the present disclosure. When such an agent is present, the ratio of cyclodextrin/active agent can be changed. A complexation-enhancing agent is a compound, or compounds, that enhance(s) the complexation of the active agent (e.g., compound of formula (I)) with the cyclodextrin. Suitable complexation enhancing agents include one or more pharmacologically inert water-soluble polymers, hydroxy acids, and other organic compounds typically used in preserved formulations to enhance the complexation of a particular agent with cyclodextrins.

Hydrophilic polymers can be used as complexation-enhancing, solubility-enhancing and/or water activity reducing agents to improve the performance of compositions containing a cyclodextrin. Suitable polymers include water-soluble natural polymers, water-soluble semi-synthetic polymers (such as the water-soluble derivatives of cellulose) and water-soluble synthetic polymers. The natural polymers include polysaccharides such as inulin, pectin, algin derivatives (e.g. sodium alginate) and agar, and polypeptides such as casein and gelatin. The semi-synthetic polymers include cellulose derivatives such as methylcellulose, hydroxyethylcellulose, hydroxypropylcellulose, their mixed ethers such as hydroxypropylmethylcellulose and other mixed ethers such as hydroxyethyl-ethylcellulose and hydroxypropylethylcellulose, hydroxypropylmethylcellulose phthalate and carboxymethylcellulose and its salts, especially sodium carboxymethylcellulose. The synthetic polymers include polyoxyethylene derivatives (polyethylene glycols) and polyvinyl derivatives (polyvinyl alcohol, polyvinylpyrrolidone and polystyrene sulfonate) and various copolymers of acrylic acid (e.g. carbomer). Other natural, semi-synthetic and synthetic polymers not named here which meet the criteria of water solubility, pharmaceutical acceptability and pharmacological inactivity are likewise considered to be within the ambit of the present disclosure. Further exemplary polymers are described, e.g., in WO 2022/093652.

As used herein, the term “stabilizer” is intended to mean a compound used to stabilize the therapeutic agent against physical, chemical, or biochemical process which would reduce the therapeutic activity of the agent. Suitable stabilizers include, by way of example and without limitation, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and other known to those of ordinary skill in the art.

As used herein, the term “tonicity modifier” is intended to mean a compound that can be used to adjust the tonicity of a liquid formulation of the composition described herein. Suitable tonicity modifiers include glycerin, lactose, mannitol, dextrose, sodium chloride, sodium sulfate, sorbitol, trehalose and others known to those of ordinary skill in the art. In some embodiments, the tonicity of the liquid formulation approximates the tonicity of blood or plasma.

In some embodiments, liquid formulations described herein include buffers and/or tonicity modifying agents to achieve properties suitable for injection into the eye. For example, in various embodiments, the pH of such liquid compositions may range from 6.0 to 8.0, 6.0 to 7.5, 6.0 to 6.8, 6.5 to 7.5, or 6.8 to 7.2. In various embodiments, the osmolality of such liquid compositions may range from 200 mOsm to 500 mOsm, 200 mOsm to 400 mOsm, or from 250 mOsm to 350 mOsm.

As used herein, the term “antifoaming agent” is intended to mean a compound that prevents or reduces the amount of foaming that forms on the surface of the liquid formulation of the composition described herein. Suitable antifoaming agents include dimethicone, simethicone, octoxynol and others known to those of ordinary skill in the art.

As used herein, the term “cryoprotectant” is intended to mean a compound used to protect an active therapeutic agent from physical or chemical degradation during lyophilization. Such compounds include, by way of example and without limitation, dimethyl sulfoxide, glycerol, trehalose, propylene glycol, polyethylene glycol, and others known to those of ordinary skill in the art.

As used herein, the term “emulsifier” or “emulsifying agent” is intended to mean a compound added to one or more of the phase components of an emulsion for the purpose of stabilizing the droplets of the internal phase within the external phase. Such compounds include, by way of example and without limitation, lecithin, polyoxylethylene-polyoxypropylene ethers, polyoxylethylene-sorbitan monolaurate, polysorbates, sorbitan esters, stearyl alcohol, tyloxapol, tragacanth, xanthan gum, acacia, agar, alginic acid, sodium alginate, bentonite, carbomer, sodium carboxymethylcellulose, cholesterol, gelatin, hydroxyethyl cellulose, hydroxypropyl cellulose, octoxynol, oleyl alcohol, polyvinyl alcohol, povidone, propylene glycol monostearate, sodium lauryl sulfate, and others known to those of ordinary skill in the art.

A solubility-enhancing agent can be added to the compositions of the present disclosure. A solubility-enhancing agent is a compound, or compounds, that enhance(s) the solubility of the active agent (e.g., compound of formula (I)) when in a liquid formulation. When such an agent is present, the ratio of cyclodextrin/active agent can be changed. Suitable solubility enhancing agents include one or more organic solvents, detergents, soaps, surfactant and other organic compounds typically used in parenteral formulations to enhance the solubility of a particular agent.

Suitable organic solvents include, for example, ethanol, glycerin, polyethylene glycols, propylene glycol, poloxamers, and others known to those of ordinary skill in the art.

Compositions of the present disclosure can include oils (e.g., fixed oils, peanut oil, sesame oil, cottonseed oil, corn oil olive oil, and the like), fatty acids (e.g., oleic acid, stearic acid, isostearic acid, and the like), fatty acid esters (e.g., ethyl oleate, isopropyl myristate, and the like), fatty acid glycerides, acetylated fatty acid glycerides, and combinations thereof. Compositions of the present disclosure can also include alcohols (e.g., ethanol, iso-propanol, hexadecyl alcohol, glycerol, propylene glycol, and the like), glycerol ketals (e.g., 2,2-dimethyl-1,3-dioxolane-4-methanol, and the like), ethers (e.g., poly(ethylene glycol) 450, and the like), petroleum hydrocarbons (e.g., mineral oil, petrolatum, and the like), water, surfactants, suspending agents, emulsifying agents, and combinations thereof.

In various embodiments of the compositions described herein, the compounds of formula (I) may be completely or substantially completely in a cis or trans configuration. In various embodiments, greater than 50%, 55%, 60%, 65%, 70%, 75%, 80%, 85%, 90%, 95%, 98%, 99%, 99.5%, or 99.9% of the molecules of Formula (I) in the composition are in the trans configuration. In various other embodiments, greater than 50%, 55%, 60%, 65%, 70%, 75%, 80%, 85%, 90%, 95%, 98%, 99%, 99.5%, or 99.9% of the molecules of Formula (I) in the composition are in the cis configuration.

In some embodiments, compositions are provided having greater than 50%, 60%, 70%, 80%, 90%, 95%, 99%, or 99.5% of the compound of Formula (I) complexed with the cyclodextrin, with the remaining amount of compound in free, un-complexed form. In some embodiments, compositions are provided having less than 90%, 80%, 70%, 60%, 50%, 40%, 30%, 20%, 10%, or 5% of the compound complexed with the cyclodextrin. In some embodiments, compositions are provided having a first percentage of compound of Formula (I) complexed with the cyclodextrin, which is then exposed to light to generate a composition having a second, lower percentage of compound of Formula (I) complexed with the cyclodextrin.

All references cited herein, including patents, patent applications, papers, textbooks and the like, and the references cited therein, to the extent that they are not already, are hereby incorporated herein by reference in their entirety.

### EXAMPLE

### Example 1. Phase 1b Clinical Study

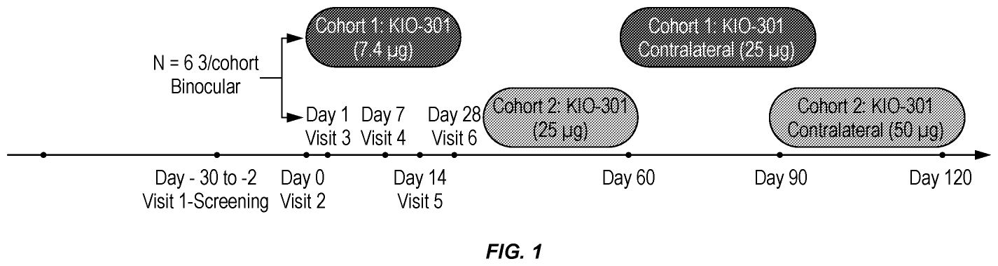

An open label, single ascending dose trial was conducted on two non-randomized cohorts. A summary of the study design is shown in FIG. 1 (assessment days shown for Cohort 1 only).

Study Design. Cohort 1 included patients with no light perception (NLP)/bare light perception (BLP). Cohort 2 included patients with counting fingers (CF)/hand motion (HM) visual acuity. The cohorts were subjected to a single intravitreal (IVT) injection of KIO-301 per eye. Primary outcome measures included adverse events (AE), pharmacokinetics (PK), and laboratory results. Secondary outcome measures included object identification, intensity, and contrast assessment, and assessment days were repeated for each cohort per eye. Representative results are discussed below.

### Results—Subject 1-01

Systemic and Ocular Safety Assessments. No AEs were reported. Slit-lamp exam revealed the subjects had abnormal baseline corneal keratopathy. Intraocular pressure (IOP) was normal at baseline. Dilated fundus photography was performed within 6 hours of injection and on Day 29 and showed abnormal baseline. Electrocardiogram was normal at baseline. No clinically significant changes from baseline for safety parameters were observed.

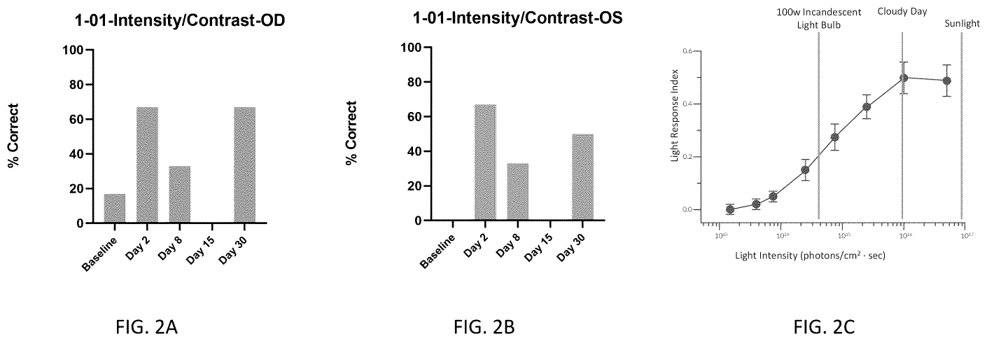

Intensity and Contrast Assessment. A series of six visual stimuli were presented to the subject, and the subject was asked to acknowledge (both verbally and physically) when a shape or object was perceived. Results are shown in FIGS. 2A-2C. FIGS. 2A and 2B show the % accuracy of the subject's perception of the shape or object with the right or left eye, respectively. FIG. 2C shows the subject's Light Response Index relative to light intensity. As shown in the results, there was a promising increase in activity from baseline to 48 hours. The subject was responsive to a low level of light (3×10¹⁴ photons/cm²·s).

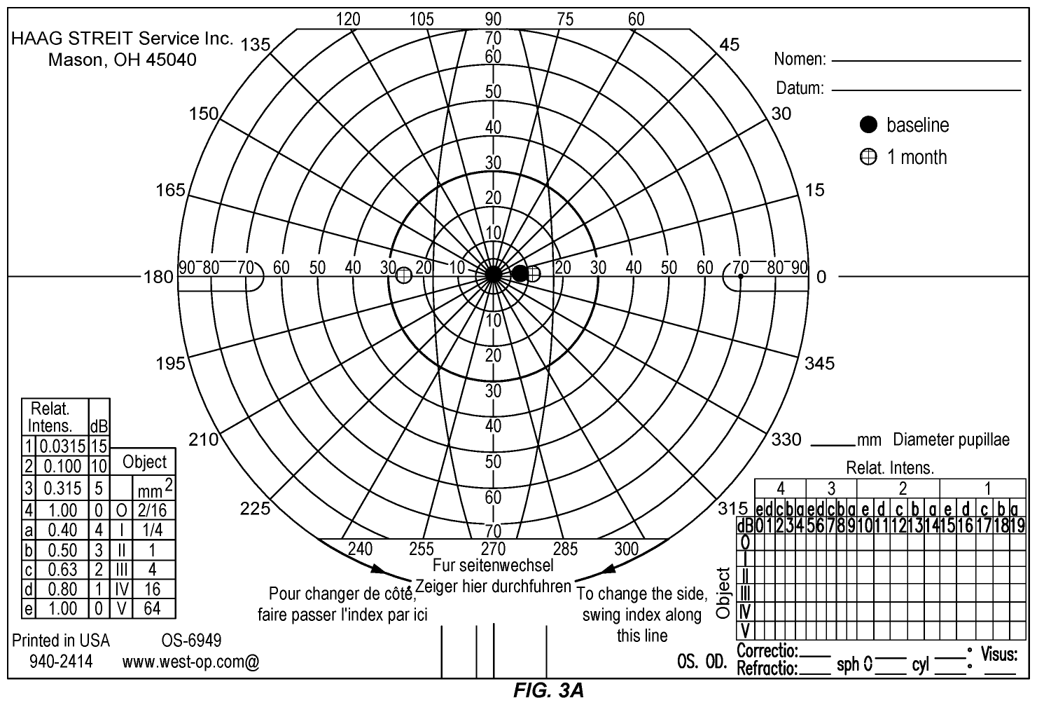

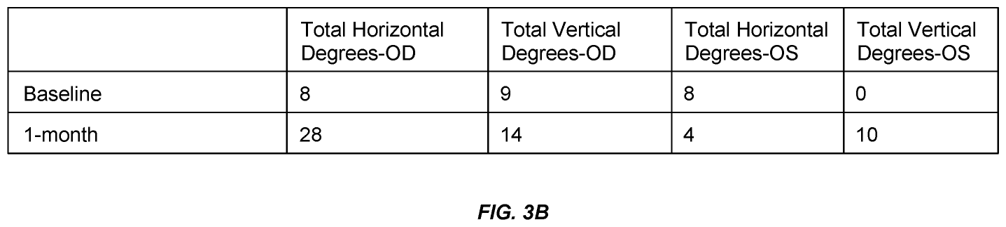

Kinetic Visual Field. Kinetic visual field was assessed with a Goldmann Haag-Streit tonometer. Assessment was conducted by an orthoptist. Total horizontal and vertical visual field degrees were measured. Results are shown in FIGS. 3A and 3B.

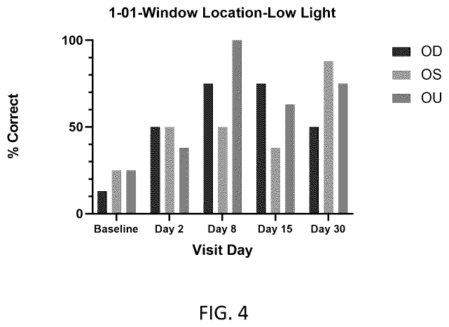

Window Location. The ability of the subject to determine direction was tested through Day 30 post-injection. The subject was asked to identify a “window” randomized to a given location 8 times. Results are shown in FIG. 4. An increasing trend for % accuracy was observed, with the effect appearing to diminish over time. The results were consistent with the subject's reported outcome.

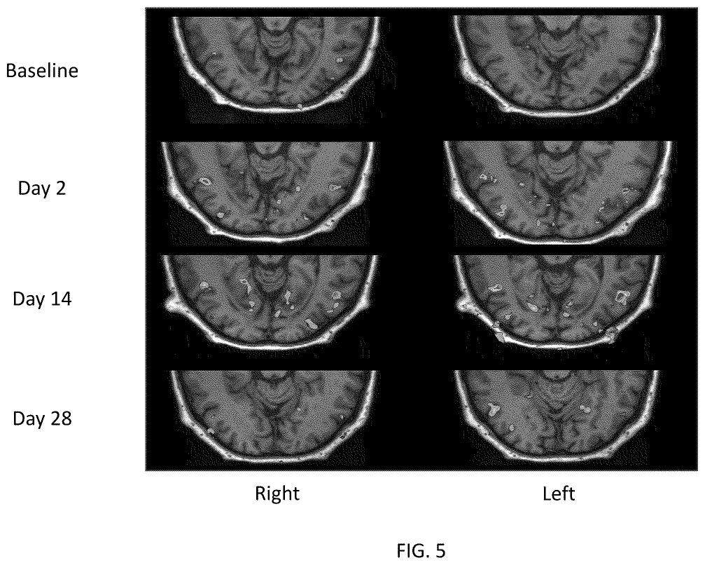

Functional MRI—Qualitative Overlap of 3 Visual Paradigms. Functional MRI was performed on the subject for baseline and Days 2, 14, and 28. Results are shown in FIG. 5. An increase in striate (V1) and non-striate (V2 and V3) activity was observed on Days 2 and 14. Day 28 activity was comparable to baseline. The bilateral response was consistent with other functional measurements of vision.

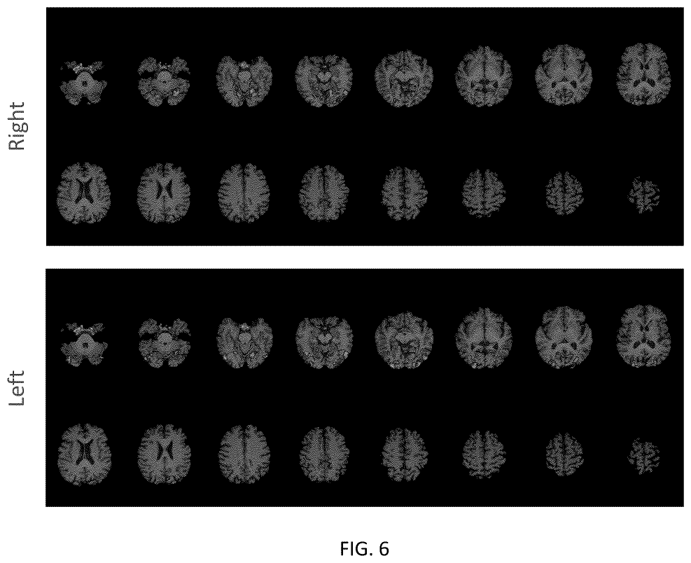

Functional MRI—Quantitative Increase: Checkerboard. Functional MRI was performed on the subject with checkerboard stimuli. Results are shown in FIG. 6. Blue areas indicate increase with p<0.05.

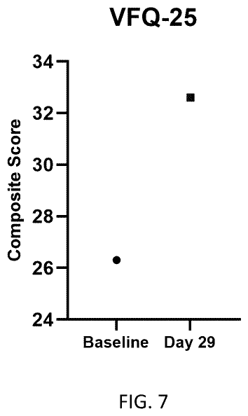

Quality of Life Survey—VFQ-25. The subject provided responses to the National Eye Institute Visual Functioning Questionnaire 25 (VFQ-25) at baseline and at Day 29 post-injection. A 2- to 4-point increase is generally accepted as clinically meaningful. Results are shown in FIG. 7. The overall increase was driven by a sub-scale increase in Vision Specific Questions (Mental Health & Role Difficulties) and Ocular Pain.

### Results—Subject 1-02.

Subject 1-02 is in the 5^{th} decade of life and has had NLP for 10+ years with an unknown genetic mutation.

Systemic and Ocular Safety Assessments. No AEs were reported. Slit-lamp exam results showed no change to baseline. Intraocular pressure (IOP) was normal at baseline and had no changes from baseline. Dilated fundus photography was abnormal at baseline and had no changes from baseline. Spectral domain optical coherence tomography (SD-OCT) showed absence of macular edema and no thinning.

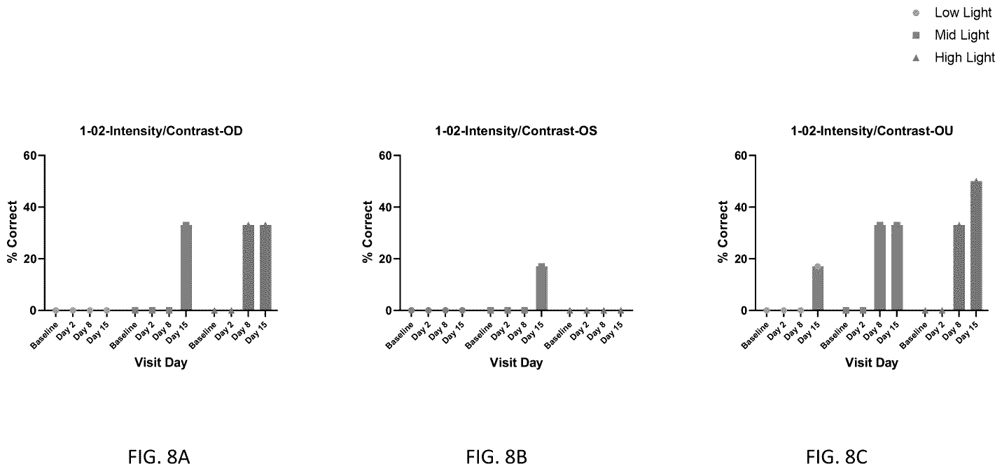

Intensity and Contrast Assessment. A series of six visual stimuli were presented to the subject at varying light levels as described above. Results are shown in FIGS. 8A-8C, demonstrating that light perception increased over the first two weeks following injection. High levels of light (5×10¹⁵ photons/cm²·s, less than a cloudy day) were perceived.

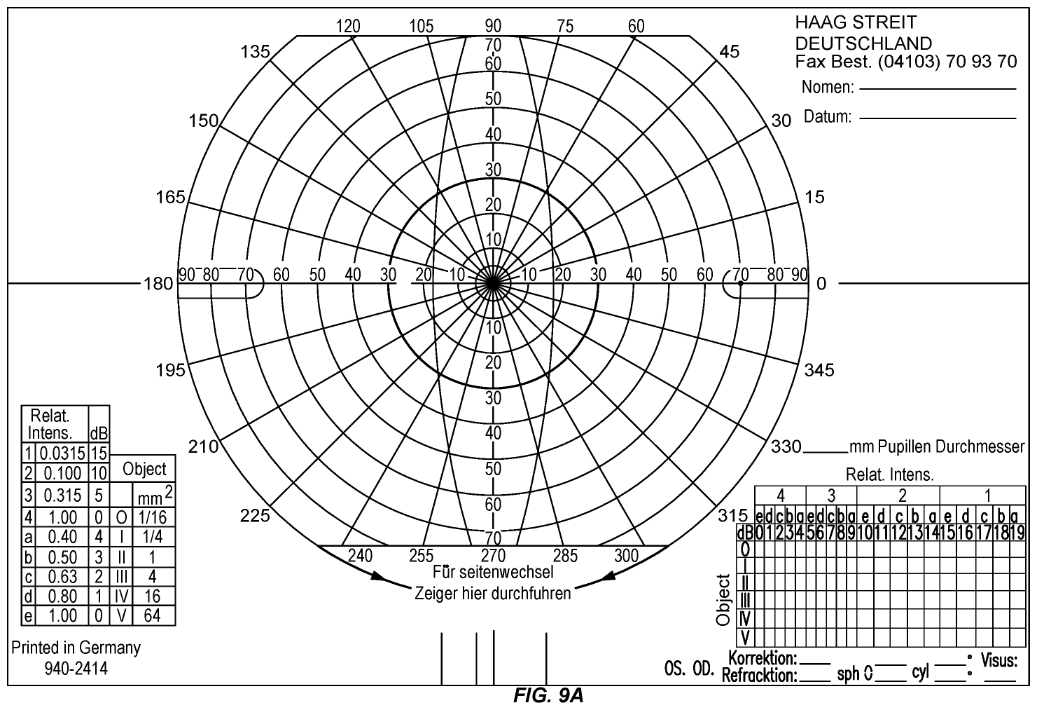

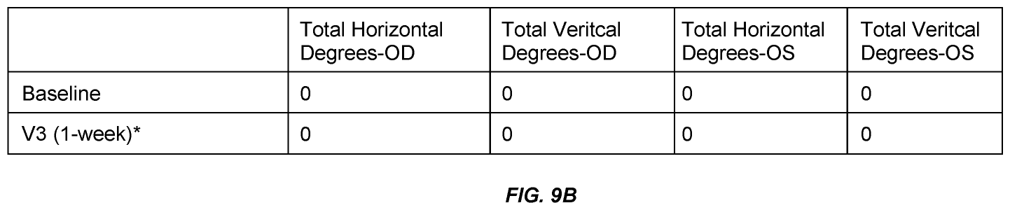

Kinetic Visual Field. Kinetic visual field was tested as described above. Results are shown in FIGS. 9A and 9B. On movement of the right eye, blue level 2 was observed in the far periphery.

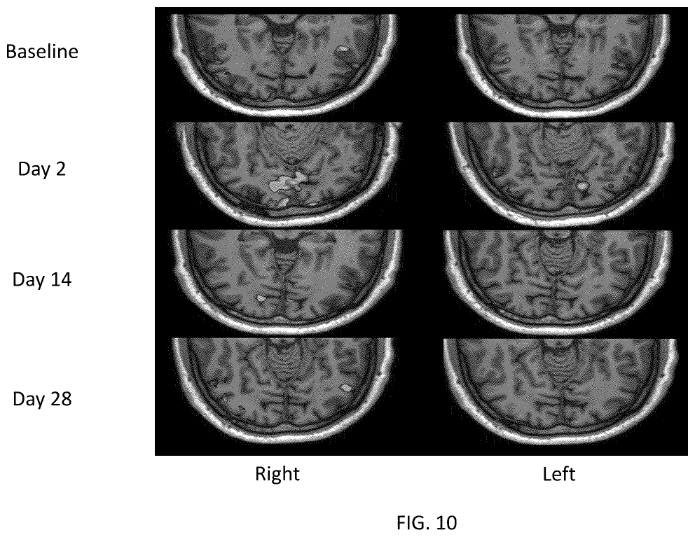

Functional MRI—Qualitative Overlap of 3 Visual Paradigms. Functional MRI was performed as described above. Results are shown in FIG. 10. A clear increase in striate (V1) activity was observed on Days 2 and 14 compared to baseline. Day 28 returned to similar activity as baseline. More unilateral increase in V1 activity was consistent with other functional measures of vision.

Subject Feedback. The subject was convinced that he perceived more light in both eyes and saw small flashes of light on a regular basis. While still not 100% clear on light/dark perception, the subject reported more confidence in his light vs. dark orientation and was able to see a lit phone screen in the dark. At 48 hours post injection during functional MRI, the subject reported consistently seeing flashes of light in the right eye upon stimuli presentation. The observations from Subject 1-02 are consistent with the kinetics of Subject 1-01, and the confirmatory functional MRI data are consistent with a stronger unilateral response.

1. A method of treating a human subject suffering from a retinal disorder, the method comprising administering to the eye of the subject via intravitreal injection a composition comprising a compound of formula (I): 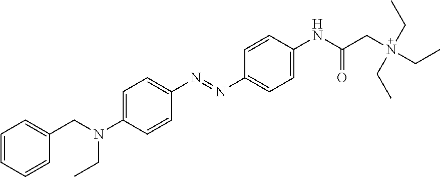
or a pharmaceutically acceptable salt thereof, wherein the azo bond in the structure may be either cis or trans.

2. The method of claim 1, wherein the retinal disorder is caused by a genetic eye disease.

3. The method of claim 2, wherein the genetic eye disease is retinitis pigmentosa.

4. The method of claim 1, wherein the retinal disorder is age-related macular degeneration.

5. The method of claim 1, wherein the retinal disorder is choroideremia.

6. The method of claim 1, wherein the compound of formula (I) is administered to both eyes of the subject.

7. The method of claim 1, wherein the compound of formula (I) is only administered to one eye of the subject.

8. The method of claim 7, wherein the administering to one eye of the subject provides a treatment effect in both eyes of the subject.

9. The method of claim 7, wherein the administering to one eye is a first administration, and wherein the method further comprises a second administration, wherein the second administration comprises administering the compound of formula (I) to contralateral eye of the subject about 1 week to about 2 months after the first administration.

10. The method of claim 7, wherein the administering to one eye is a first administration, and wherein the method further comprises a second administration, wherein the second administration comprises administering the compound of formula (I) to the same eye or the collateral eye of the subject about 1 week to about 2 months after the first administration.

11. (canceled)

12. The method of claim 7, wherein the administering to one eye is a first administration, and wherein the method further comprises a second administration, wherein the second administration comprises administering the compound of formula (I) to the same eye or the collateral eye of the subject about 3 weeks to 5 weeks after the first administration.

13. (canceled)

14. The method of claim 7, wherein the administering to one eye is a first administration, and wherein the method further comprises a second administration, wherein the second administration comprises administering the compound of formula (I) to the same eye or the collateral eye of the subject about 1 month to about 2 months after the first administration.

15. The method of claim 1, wherein the treating a human subject comprises increasing the subject's ability to detect light and contrast as measured by a light response test.

16. The method of claim 1, wherein the treating a human subject comprises stimulating striate (V1) and non-striate (V2 and/or V3) cortical activity in the subject as measured by Functional MRI.

17. The method of claim 1, wherein the treating a human subject comprises an increase in tolerance of glare and photosensitivity in the subject.

18. The method of claim 1, wherein the treating of a human subject comprises increasing measures of functional vision.

19. The method of claim 1, wherein the treating of a human subject reduces the non-specific, non-stimulus generated, random electrical activity of the retina.

20. The method of claim 1, wherein the composition further comprises a cyclodextrin.

21. The method of claim 20, wherein a molar ratio of cyclodextrin to the compound of Formula I in the composition is about 20:1 to about 1:1.

22. (canceled)

23. (canceled)

24. The method of claim 1, wherein the concentration of the compound of formula (I) in the composition is from about 0.1 mM to about 10 mM.

25. (canceled)

26. The method of claim 1, wherein the human subject is administered about 1 μg to about 100 μg of the compound of formula (I) to a single eye.

27. (canceled)

28. A method of treating a human subject suffering from a retinal disorder, the method comprising:
a. a first administration to one eye of the subject via intravitreal injection a composition comprising a compound of formula (I); and a second administration to the contralateral eye of the subject via intravitreal injection a composition comprising a compound of formula (I); or
b. a first administration to one eye of the subject via intravitreal injection a composition comprising a compound of formula (I); and a second administration to the same eye of the subject via intravitreal injection a composition comprising a compound of formula (I);
wherein the compound of formula (I) has the structure: 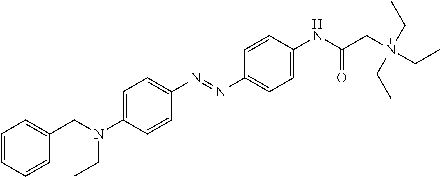
or a pharmaceutically acceptable salt thereof, wherein the azo bond in the structure may be either cis or trans.

29. The method of claim 28, wherein the second administration to the contralateral eye is simultaneously or substantially simultaneously as the first administration, or immediately following the first administration.

30. The method of claim 28, wherein the second administration to the contralateral eye is about 3 weeks to about 5 weeks after the first administration.

31. The method of claim 28, wherein the second administration to the same eye is about 3 weeks to about 5 weeks after the first administration.

32. A method of increasing the tolerance from glare and photosensitivity in a human subject suffering from a retinal disorder, the method comprising:
a. a first administration to one eye of the subject via intravitreal injection a composition comprising a compound of formula (I); and a second administration to the contralateral eye of the subject via intravitreal injection a composition comprising a compound of formula (I); or
b. a first administration to one eye of the subject via intravitreal injection a composition comprising a compound of formula (I); and a second administration to the same eye of the subject via intravitreal injection a composition comprising a compound of formula (I);
wherein the compound of formula (I) has the structure: 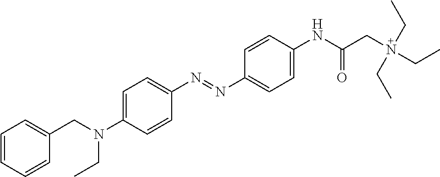
or a pharmaceutically acceptable salt thereof, wherein the azo bond in the structure may be either cis or trans.
